(19) United States
(12) Patent Application Publication
(10) Pub. No.: US 2008/0267903 A1
(43) Pub. Date: Oct. 30, 2008

(54) Bioactive Polymers

(76) Inventors: Ijeoma Uchegbu, St. Albans (GB); Andreas G. Schatzlein, St. Albans (GB); Christine Dufes, Glasgow (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/577,339

(22) PCT Filed: Oct. 14, 2005
(86) PCT No.: PCT/GB05/03976

§ 371 Date: Mar. 11, 2008

(30) Foreign Application Priority Data
Oct. 14, 2004 (GB) 0422877.1

(51) Int. Cl. A61K 31/785; A61P 35/00

(52) U.S. Cl. 424/78.35; 424/78.08

(57) ABSTRACT

Various polymers, including cationic polyamine polymers and dendrimeric polymers, are shown to possess anti-proliferative activity, and may therefore be useful for treatment of disorders characterised by undesirable cellular proliferation such as neoplasms and tumours, inflammatory disorders (including autoimmune disorders), psoriasis and atherosclerosis. The polymers may be used alone as active agents, or as delivery vehicles for other therapeutic agents, such as drug molecules or nucleic acids for gene therapy. In such cases, the polymers' own intrinsic anti-tumour activity may complement the activity of the agent to be delivered.

### FIELD OF THE INVENTION

This invention relates to bioactive polymer compounds, including oligomer and dendrimer compounds, pharmaceutical compositions comprising such compounds, and the use of such compositions and compounds to treat various conditions alleviated by the inhibition, reduction or control of unwanted or undesirable cellular proliferation.

### BACKGROUND TO THE INVENTION

Despite the number of deaths from cancer in 2000 being lower than estimated in 1985 cancer remains a leading cause of death in Europe [1]. In addition to the suffering and distress for patients and their families, the treatment of cancer clearly poses an enormous public health problem with wide ranging socioeconomic implications.

Currently therapeutic options are limited and only 4% of patients requiring systemic treatment can be cured. The idea of a drug as the magic bullet, originally suggested at the end of the 19th century by Nobel Laureate Paul Ehrlich, has since provided the paradigm for drug targeting. Pharmacologists have striven to develop so-called ‘clean’ drugs that avoid the sometimes dramatic and even life-threatening side effects of anticancer therapy often synonymous with ‘chemotherapy’ in the public's mind. A good example of this is alopecia induced by chemotherapy. This is an obvious side-effect with significant associated psychosocial morbidity; directing the drug away from the hair follicle would thus represent a significant therapeutic improvement. Over the years, improved administration modalities and novel cytotoxic drugs have led to significant improvements in the management of cancer [1, 2]. However, the need for safe and efficacious drugs to treat various forms of cancer remains high.

Cationic polyamine polymers (CPPs) have previously been used in various ways in biomedical research and pharmaceutical products, mainly as excipients in pharmaceutical formulations, but also to assist in delivery of drug molecules, gene delivery vectors, or other biomedical materials.

Naturally occurring polyamines (putrescine, spermidine, and spermine) play multifunctional roles in cell growth and differentiation but recently have also been implicated in promoting apoptosis [3]. Analogues of these natural polyamines have been developed as potential anti-cancer agents. These analogues include N1,N11-diethylnorspermine [4]. Various conformationally restricted and/or unsaturated synthetic polyamines, including analogues of 1N,12N-bisethylspermine, ¹N,¹⁴N-Bisethylhomospermine (BE-4-4-4), and 3,8,13,18,23-pentaazapentacosane (BE-4-4-4-4), have also been investigated for anti-cancer activity [5, 6, 7, 8, 9].

Frydman and colleagues report activity of the polyamine analogue SL-11093 (3,8,13,18-tetraaza-10,11-[(E)-1,2-cyclopropyl]eicosane tetrahydrochloride) against xenografts in mouse models [10]. A series of cyclopropane containing analogues have been shown to be active in xenograft models [11,12].

Liu and colleagues [13] review the effect of heparin-like glycosaminoglycans in tumour biology and report that these molecules can promote or inhibit tumour growth. Berry et al. [14] report that in cell culture the heparan sulfate-like glycosaminoglycans, and in particular heparin, were able to induce apoptosis of cancer cells when internalised. They also report that some members of a library of poly(beta-amino ester)s internalize heparin and thus inhibit tumour cell growth by up to 73% [14] but they do not show that these compounds behave any differently towards tumour cells and healthy cells, or demonstrate therapeutic applicability. Furthermore Ishida and colleagues report the effects of heparin sulphate glycosaminoglycans mimetic compounds may exert an anti-cancer effect, but suggest that this is due to increased adherence of the cells, rather than by uptake of the polymers [15].

Dendrimer compounds have variously been used for delivery of a bioactive agent. Many of the biomedical and pharmaceutical application of dendrimers focus on PAMAM dendrimers [16-19], gene delivery [20-27] and phosphorous containing [28] compounds with a mixture of amine/amide or N—P(O2)S as the conjugating units respectively. Polypropylenimine dendrimers have also been studied as pH-sensitive controlled release systems for drug delivery [29, 30] and for their encapsulation of guest molecules when chemically modified by peripheral amino acid groups [31]. Previous patent applications describing dendrimers (e.g. for as delivery agents) include U.S. Pat. No. 5,714,166, U.S. Pat. No. 5,990,089, U.S. Pat. No. 5,795,581 and WO03/001218.

Kabanov and others report that polypropylenimine dendrimers interact with DNA via the surface primary amines only with no involvement of the internal amine groups [33] while Gebhart and Kabanov report very low gene transfer activity with the 5th generation polypropylenimine dendrimers DAB 64 in the easy-to-transfect COS cell line [34] and conclude that DAB 64 is far too toxic above a dendrimer-DNA weight ratio of 0.62:1 (nitrogen to phosphate ratio of 4:1). Additionally Malik and others concluded that the cationic dendrimers as opposed to the anionic dendrimers are too toxic for parenteral use without further derivatisation with biocompatible groups such as polyethylene glycol units [35].

The present inventors have recently demonstrated that the lower generation PPI dendrimers strike a favourable balance between their ability to transfect and their cytotoxicity [36, 37] and can also be used to deliver oligonucleotides into cells [38]; see also WO03/033027.

Duncan and colleagues describe the use of anionic PAMAM dendrimers coupled to a cytotoxic agent, such as a platinum containing compound (U.S. Pat. No. 6,585,956). Shaunak et al. describe an anionic (generation 3.5) PAMAM dendrimer conjugated to glucosamine and (separately) to glucosamine-6-sulfate, the glucosamine compounds having previously been reported to improve wound healing. The glucosamine and glucosamine-6-sulfate conjugates are reported to prevent scar tissue formation, but the non-conjugated dendrimer was found to have no biological activity of its own. The anionic, carboxyl-terminated, dendrimer was chosen because of its purported lack of toxicity compared to cationic amine-terminated PAMAM dendrimers [59]. Gong et al. report antiviral activities exhibited by a polyanionic lysine dendrimer, SPL-2999, in which the surface (terminal) groups are sodium salts of naphthyl 3,6-disulfonic acid [60].

Polyethylenimine (PEI) polymers have been extensively used as gene delivery agents in vitro and in vivo [40]. Most of the PEI formulations studied to date have been prepared using branched PEI of varying molecular weight (0.6 kD-800 kD), but a linear PEI of 22 kD has also been examined. Polyplexes from higher MW branched PEIs (70-800 kD) were found to be more efficient in vitro [40-43] but on intravenous administration the smaller and linear PEIs [44, 45] seem in general to be more efficient than branched PEI of 25 kD PEI [46, 47] or 50-750 kD PEI [48, 49]. More recently, cholesteryl PEI derivatives have also been shown to transfect cells [50, 51]. Targeted PEI based DNA complexes have been used to delivery genes to tumour xenografts [52], but the authors did not identify any specific antitumour activity provided by the polymer itself.

Brownlie et al. describe a number of modifications of branched PEI but do not report any activity from the polymer itself [53].

### SUMMARY OF THE INVENTION

The present inventors have found that certain cationic polymers have highly selective antiproliferative properties in vivo, which makes them particularly suitable for use as therapeutic agents for the treatment of diseases characterised by undesirable cellular proliferation. A number of these cationic polymers have previously been used to deliver agents such as nucleic acid into target cells, but their potential as therapeutic agents in their own right has, until now, been unrecognised.

A first aspect of the present invention is the use of a compound of formula I or a salt thereof as an active agent in the preparation of a medicament for the treatment of a condition characterised by undesirable cellular proliferation:

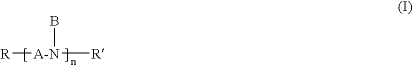

wherein
R is independently selected from H, optionally substituted C₁₋₁₆ alkyl and NR²R³ wherein R² and R³ are independently selected from H and optionally substituted C₁₋₁₆ alkyl;
R′ is independently selected from H and optionally substituted C₁₋₁₆ alkyl;
n denotes the number of backbone monomer units -[A-N(B)]- and is greater than or equal to 15;
the A groups of the backbone monomer units are independently selected from optionally substituted C₁₋₁₆alkylene groups; and
the B groups of the backbone monomer units are independently selected from H, optionally substituted C₁₋₁₆ alkyl and a branching group of formula II:

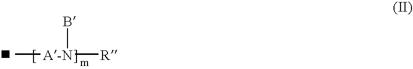

wherein
R″ is selected from H, optionally substituted C₁₋₁₆ alkyl and optionally substituted C₁₋₁₆ alkylene-NR²R³;
m denotes the number of monomer units -[A′-N(B′)]— of the branching group and is greater than or equal to 1;
the A′ groups of the monomer units of the branching group are independently selected from optionally substituted C₁₋₁₆ alkylene groups; and
the B′ groups of the monomer units of the branching group are independently selected from H, optionally substituted C₁₋₁₆ alkyl and a branching group of formula II;
wherein each of said C₁₋₁₆ alkyl and C₁₋₁₆ alkylene groups is optionally interrupted by one or more N(R²) or O heterogroups.

A second aspect of the invention is the use of a dendrimer compound of the general formula III or a salt thereof as an active agent in the preparation of a medicament for the treatment of a condition characterised by undesirable cellular proliferation:

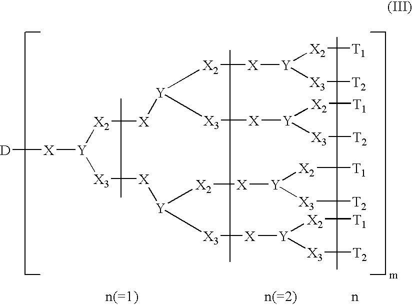

wherein
n is greater than or equal to 1, wherein n represents the number of generations of the dendrimer;
D is a core group of the dendrimer including a plurality of functional atoms;
Y is selected independently for each generation of the dendrimer from N or C(R¹) wherein each R¹ is independently H or optionally substituted C₁₋₆ alkyl;
X, X₂ and X₃ are independently selected, independently for each generation of the dendrimer, from a single bond, optionally substituted C₁₋₁₆alkylene groups, and N(R²), wherein each R² is independently H or optionally substituted C₁₋₁₆ alkyl, and wherein said C₁₋₁₆ alkyl and C₁₋₁₆ alkylene groups are independently optionally interrupted by one or more N(R²) or O heterogroups;
m is an integer from 2 to 8, wherein m denotes the number of X groups of the first generation that are bonded to the core group, wherein each X group of the first generation is bonded to a core functional atom; and
T₁ and T₂ represent end groups bonded to the nth generation of the dendrimer, wherein T₁ and T₂ are independently selected from the substituents defined herein.

While certain dendrimer compounds falling within Formula III have previously been used for delivery of therapeutic agents such as nucleic acids, they have not previouly been suggested for use as therapeutic agents in their own right. The compound of formula III, or salt thereof, may therefore be used in a composition (such as a pharmaceutical composition) as the sole active agent present. Thus, in some embodiments, the composition does not contain nucleic acid or other therapeutic agent which is active for the treatment of a condition characterized by undesirable cellular proliferation (e.g. a cytotoxic agent) in a therapeutically effective amount; for example, the composition may not contain nucleic acid or other therapeutic agent at all.

In alternative embodiments, other active agents may be present, but need not be complexed with the dendrimer compound of formula III. Thus the compound of formula III or salt thereof is preferably not complexed to a nucleic acid molecule or other therapeutic agent which is active for the treatment of a condition characterized by undesirable cellular proliferation (e.g. a cytotoxic agent).

Certain polymers having previously unrecognised antiproliferative properties may be used as delivery agents for other therapeutic agents such as cytotoxic drugs. A third aspect of the present invention is therefore a composition for delivering a bioactive molecule other than a nucleic acid to a target location in vivo, the composition comprising a compound of formula I or a salt thereof admixed with said bioactive molecule, wherein the composition does not contain nucleic acid:

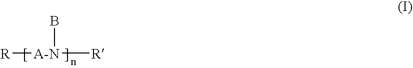

wherein
R is independently selected from H, optionally substituted C₁₋₁₆ alkyl and NR²R³ wherein R² and R³ are independently selected from H and optionally substituted C₁₋₁₆ alkyl;
R′ is independently selected from H and optionally substituted C₁₋₁₆ alkyl;
n denotes the number of backbone monomer units -[A-N(B)]- and is greater than or equal to 3;
the A groups of the backbone monomer units are independently selected from optionally substituted C₁₋₁₆ alkylene groups; and
the B groups of the backbone monomer units are independently selected from H, optionally substituted C₁₋₁₆ alkyl and a branching group of formula II:

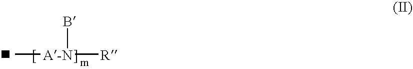

wherein
R″ is selected from H, optionally substituted C₁₋₁₆ alkyl and optionally substituted C₁₋₁₆ alkylene-NR²R³;
m denotes the number of monomer units -[A′-N(B′)]— of the branching group and is greater than or equal to 1;
the A′ groups of the monomer units of the branching group are independently selected from optionally substituted C₁₋₁₆ alkylene groups; and
the B′ groups of the monomer units of the branching group are independently selected from H, optionally substituted C₁₋₆ alkyl and a branching group of formula II;
wherein each of said C₁₋₁₆ alkyl and C₁₋₁₆ alkylene groups is optionally interrupted by one or more N(R²) or O heterogroups.

Such compositions typically contain small complexes formed between the cationic polymer and the bioactive molecule. The complexes may take the form of small “nanoparticles”. For optimal complex formation, the bioactive molecule is preferably anionic, and preferably carries more than one negative charge per molecule, in order that the cationic groups of the polymer are able to form non-covalent electrostatic interactions with the bioactive molecule.

The compositions of this aspect of the invention may be particularly therapeutically effective because both the bioactive molecule and the polymer have therapeutic (e.g. antitumour) activity in their own right. Thus the compositions may provide an additive or even synergistic antiproliferative effect, in excess of the effect which would be obtained using the bioactive molecule alone.

A further aspect of the present invention provides the use of a composition as described in relation to the third aspect of the invention, or a pharmaceutically acceptable derivative thereof, in the preparation of a medicament for the treatment of a condition characterised by undesirable cellular proliferation.

Another aspect of the present invention provides a method of treating a condition characterised by undesirable cellular proliferation, which method comprises administering to a patient in need of treatment an effective amount of a compound of formula I or III, or a composition according to the third aspect of the invention, or a pharmaceutically acceptable derivative or salt thereof.

Another aspect of the present invention provides novel compounds or salts, solvates and chemically protected forms thereof, and methods of synthesis thereof as described herein.

Conditions which may be treated by the compounds and compositions described herein include conditions characterised by undesirable cellular proliferation, that is to say, conditions characterised by an unwanted or undesirable proliferation of normal or abnormal cells. Such conditions may involve neoplastic or hyperplastic growth of any type of cell, or inflammatory or autoimmune disorders in which proliferation of cells of the immune system gives rise to tissue damage or other symptoms of disease, which may be caused by direct cellular activity or by mediators released by the cells of the immune system.

Examples of conditions characterised by undesirable cellular proliferation include, but are not limited to, benign, pre-malignant, and malignant cellular proliferation, including but not limited to, neoplasms and tumours (e.g., histocytoma, glioma, astrocytoma, osteoma), cancers (e.g., lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), leukemias, psoriasis, bone diseases, fibroproliferative disorders (e.g., of connective tissues), atherosclerosis and inflammatory disorders.

Thus the compounds and compositions described herein may be useful in the treatment of chronic autoimmune conditions and/or inflammation (including, for example, rheumatoid arthritis); in the therapeutic and/or preventative treatment of localised lesions; for inhibiting angiogenesis (e.g. in the treatment of solid tumours); and in the treatment of wound healing (e.g. to reduce unwanted scar tissue formation, for example in relation to operations or burn injuries). Thus, the compounds and compositions described herein may be useful for preventing or reducing scar tissue formation during angioplasties (and may therefore be suitable for drug-coating stents for use in such procedures). The compounds and compositions described herein may also be useful for preventing the formation of unwanted tissue and vascularisation in the eye, e.g. in the cornea.

### DEFINITIONS

Oxo (keto, -one): The term “oxo”, as used herein, pertains to the monovalent moiety ═O, also known as a keto group.

Halo: The term “halo”, as used herein, pertains to the monovalent moiety —Y, wherein Y is a halogen atom. Examples of halo groups include —F, —Cl, —Br, and —I.

Hydroxy: The term “hydroxy”, as used herein, pertains to the monovalent moiety —OH.

Carboxy (carboxylic acid): The term “carboxy”, as used herein, pertains to the monovalent moiety —C(═O)OH.

Alkyl: The term “alkyl,” as used herein, pertains to a monovalent moiety obtained by removing a hydrogen atom from a carbon atom of a hydrocarbon compound having from 1 to 16 carbon atoms (unless otherwise specified), which may be aliphatic or alicyclic, and which may be saturated or unsaturated (e.g., partially unsaturated, fully unsaturated). Thus, the term “alkyl” includes the sub-classes alkenyl, alkynyl, cycloalkyl, cycloalkyenyl, cylcoalkynyl, etc.

In the context of alkyl groups, the prefixes (e.g., C₁₋₄, C₁₋₆, C₁₋₁₆, C₂₋₇, C₃₋₇, etc.) denote the number of carbon atoms, or range of number of carbon atoms. For example, the term “C₁₋₆alkyl,” as used herein, pertains to an alkyl group having from 1 to 16 carbon atoms. Examples of groups of alkyl groups include C₁₋₄ alkyl (“lower alkyl”), C₁₋₆alkyl, C₁₋₁₂ alkyl and C₁₋₁₆alkyl. Note that the first prefix may vary according to other limitations; for example, for unsaturated alkyl groups, the first prefix must be at least 2; for cyclic alkyl groups, the first prefix must be at least 3; etc.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄) pentadecyl (C₁₅) and hexadecyl (C₁₆).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇) .

Examples of (unsubstituted) saturated branched alkyl groups include iso-propyl (C₃), iso-butyl (C₄), sec-butyl (C₄), tert-butyl (C₄), iso-pentyl (C₅), and neo-pentyl (C₅).

Cycloalkyl: The term “cycloalkyl”, as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 ring atoms (unless otherwise specified).

Examples of saturated cycloalkyl groups include, but are not limited to, those derived from: cyclopropane (C₃), cyclobutane (C₄), cyclopentane (C₅), cyclohexane (C₆), cycloheptane (C₇), norbornane (C₇), norpinane (C₇), norcarane (C₇).

Alkenyl: The term “alkenyl,” as used herein, pertains to an alkyl group having one or more carbon-carbon double bonds. Examples of groups of alkenyl groups include C₂₋₄ alkenyl, C₂₋₇ alkenyl, C₂₋₂₀ alkenyl.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (—C(CH₃)═CH₂), butenyl (C₄), pentenyl (C₅), and hexenyl (C₆).

Examples of unsaturated cyclic alkenyl groups, which are also referred to herein as “cycloalkenyl” groups, include, but are not limited to, cyclopropenyl (C₃), cyclobutenyl (C₄), cyclopentenyl (C₅), and cyclohexenyl (C₆).

Heterocyclyl: The term “heterocyclyl,” as used herein, pertains to a monovalent moiety obtained by removing a hydrogen atom from a ring atom of a heterocyclic compound, which moiety has from 3 to 7 ring atoms, of which from 1 to 4 are ring heteroatoms.

In this context, the prefixes (e.g., C₃₋₇, C₅₋₆, etc.) denote the number of ring atoms, or range of number of ring atoms, whether carbon atoms or heteroatoms. For example, the term “C₃₋₇ heterocyclyl,” as used herein, pertains to a heterocyclyl group having 3, 4, 5, 6 or 7 ring atoms. Examples of groups of heterocyclyl groups include C₃₋₇heterocyclyl, C₅₋₇heterocyclyl, and C₅₋₆heterocyclyl.

Examples of (non-aromatic) monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of heterocyclyl groups which are also heteroaryl groups are described below with aryl groups.

Aryl: The term “aryl,” as used herein, pertains to a monovalent moiety obtained by removing a hydrogen atom from an aromatic ring atom of an aromatic compound, which moiety has from 5 to 10 ring atoms (unless otherwise specified). Preferably, each ring has from 5 to 7 ring atoms, more preferably, from 5 to 6 ring atoms.

In this context, the prefixes (e.g., C₅₋₁₀, C₅₋₇, C₅₋₆, etc.) denote the number of ring atoms, or range of number of ring atoms, whether carbon atoms or heteroatoms. For example, the term “C₅₋₆ aryl,” as used herein, pertains to an aryl group having 5 or 6 ring atoms. Examples of groups of aryl groups include C₃₋₁₀aryl, C₅₋₁₀aryl, C₅₋₇aryl, C₅₋₆aryl, C₅aryl, and C₆aryl.

The ring atoms may be all carbon atoms, as in “carboaryl groups.” Examples of carboaryl groups include C₅₋₁₀carboaryl, C₅₋₇carboaryl, C₅₋₆carboaryl, C₅carboaryl, and C₆carboaryl.

Examples of carboaryl groups include, but are not limited to, those derived from benzene (i.e., phenyl) (C₆), naphthalene (C₁₀), and azulene (C₁₀).

Examples of aryl groups which comprise fused rings, at least one of which is an aromatic ring, include, but are not limited to, groups derived from indane (e.g., 2,3-dihydro-1H-indene) (C₉), indene (C₉), isoindene (C₉), and tetraline (1,2,3,4-tetrahydronaphthalene) (C₁₀).

Alternatively, the ring atoms may include one or more heteroatoms, as in “heteroaryl groups.” Examples of heteroaryl groups include C₅₋₁₀heteroaryl, C₅₋₇heteroaryl, C₅₋₆heteroaryl, C₅heteroaryl, and C₆heteroaryl.

Examples of monocyclic heteroaryl groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heterocyclic groups (some of which are also heteroaryl groups) which comprise fused rings, include, but are not limited to:
C₉ heterocyclic groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀ heterocyclic groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄).

Heterocyclic groups (including heteroaryl groups) which have a nitrogen ring atom in the form of an —NH— group may be N-substituted, that is, as —NR—. For example, pyrrole may be N-methyl substituted, to give N-methylpyrrole. Examples of N-substitutents include, but are not limited to C₁₋₇alkyl, C₃₋₂₀heterocyclyl, C₅₋₂₀aryl, and acyl groups.

Heterocyclic groups (including heteroaryl groups) which have a nitrogen ring atom in the form of an —N=group may be substituted in the form of an N-oxide, that is, as —N(→O)=(also denoted —N⁺(→O⁻)═). For example, quinoline may be substituted to give quinoline N-oxide; pyridine to give pyridine N-oxide; benzofurazan to give benzofurazan N-oxide (also known as benzofuroxan).

Cyclic groups may additionally bear one or more oxo (═O) groups on ring carbon atoms.

Amino: —NR¹R², wherein R¹ and R² are independently amino substituents, for example, hydrogen, a C₁₋₁₆alkyl group (also referred to as C₁₋₁₆ alkylamino or di-C₁₋₁₆ alkylamino), a C₃₋₇ heterocyclyl group, or a C₅₋₇ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Alkylene: The term “alkylene,” as used herein, pertains to a bidentate moiety obtained by removing two hydrogen atoms, either both from the same carbon atom, or one from each of two different carbon atoms, of a hydrocarbon compound having from 1 to 16 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc.

In this context, the prefixes (e.g., C₁₋₄, C₁₋₆, C₁₋₁₆, C₂₋₇, C₃₋₇, etc.) denote the number of carbon atoms, or range of number of carbon atoms. For example, the term “C₁₋₁₆alkylene,” as used herein, pertains to an alkylene group having from 1 to 16 carbon atoms. Examples of groups of alkylene groups include C₁₋₄ alkylene (“lower alkylene”), C₁₋₆alkylene, and C₁₋₁₂ alkylene.

Examples of linear saturated C₁₋₁₆alkylene groups include, but are not limited to, —(CH₂)ₙ— where n is an integer from 1 to 12, for example, —CH₂— (methylene), —CH₂CH₂— (ethylene), —CH₂CH₂CH₂-(propylene), —CH₂CH₂CH₂CH₂— (butylene), —CH₂CH₂CH₂CH₂CH₂CH₂-(hexylene), —CH₂CH₂CH₂CH₂CH₂CH₂CH₂CH₂CH₂CH₂CH₂CH₂— (dodecylene) and —CH₂CH₂CH₂CH₂CH₂CH₂CH₂CH₂CH₂CH₂CH₂CH₂CH₂CH₂CH₂CH₂— (hexadecylene).

Examples of branched saturated C₁₋₆ alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₁₋₆ alkylene groups include, but is not limited to, —CH═CH— (vinylene), —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, and —CH═CH—CH₂—CH₂—CH═CH—.

Examples of branched partially unsaturated C₁₋₆ alkylene groups include, but is not limited to, —C(CH₃)═CH—, —C(CH₃)═CH—CH₂—, and —CH═CH—CH(CH₃)—.

Examples of alicyclic saturated C₁₋₆ alkylene groups include, but are not limited to, cyclopentylene (e.g., cyclopent-1,3-ylene), and cyclohexylene (e.g., cyclohex-1,4-ylene).

Examples of alicyclic partially unsaturated C₁₋₆ alkylene groups include, but are not limited to, cyclopentenylene (e.g., 4-cyclopenten-1,3-ylene), cyclohexenylene (e.g., 2-cyclohexen-1,4-ylene; 3-cyclohexen-1,2-ylene; 2,5-cyclohexadien-1,4-ylene).

Arylene: The term “arylene,” as used herein, pertains to a bidentate moiety obtained by removing two hydrogen atoms, one from each of two different aromatic ring atoms of an aromatic compound, which moiety has from 5 to 10 ring atoms (unless otherwise specified). Preferably, each ring has from 5 to 7 ring atoms, more preferably from 5 to 6 atoms.

In this context, the prefixes (e.g., C₅₋₁₀, C₅₋₇, C₅₋₆, etc.) denote the number of ring atoms, or range of number of ring atoms, whether carbon atoms or heteroatoms. For example, the term “C₅₋₆arylene,” as used herein, pertains to an arylene group having 5 or 6 ring atoms. Examples of groups of arylene groups include C₅₋₁₀arylene, C₅₋₇arylene, C₅₋₆arylene, C₅arylene, and C₆arylene.

The ring atoms may be all carbon atoms, as in “carboarylene groups” (e.g., C₅₋₁₀carboarylene).

Examples of C₅₋₁₀arylene groups which do not have ring heteroatoms (i.e., C₅₋₁₀ carboarylene groups) include, but are not limited to, those derived from the compounds discussed above in regard to carboaryl groups.

Alternatively, the ring atoms may include one or more heteroatoms, as in “heteroarylene groups” (e.g., C₅₋₁₀ heteroarylene).

Examples of C₅₋₁₀ heteroarylene groups include, but are not limited to, those derived from the compounds discussed above in regard to heteroaryl groups.

Arylene-alkylene: The term “arylene-alkylene,” as used herein, pertains to a bidentate moiety comprising an arylene moiety, -Arylene-, linked to an alkylene moiety, -Alkylene-, that is, -Arylene-Alkylene-.

Examples of arylene-alkylene groups include, e.g., C₅₋₁₀arylene-C₁₋₁₆alkylene, such as, for example, phenylene-methylene, phenylene-ethylene, phenylene-propylene, and phenylene-ethenylene (also known as phenylene-vinylene).

Alkylene-arylene: The term “alkylene-arylene,” as used herein, pertains to a bidentate moiety comprising an alkylene moiety, -Alkylene-, linked to an arylene moiety, -Arylene-, that is, -Alkylene-Arylene-.

Examples of alkylene-arylene groups include, e.g., C₁₋₁₆alkylene-C₅₋₁₀arylene, such as, for example, methylene-phenylene, ethylene-phenylene, propylene-phenylene, and ethenylene-phenylene (also known as vinylene-phenylene).

Alkylene and alkyl groups may be “optionally interrupted” by one or more N(R) heterogroups or O heteroatoms.

The phrase “optionally interrupted”, as used herein, pertains to an alkyl or alkylene group, as above, which may be uninterrupted or which may be interrupted by a multivalent heteroatom such as boron, silicon, nitrogen, phosphorus, oxygen, sulfur, and selenium (more commonly nitrogen and oxygen).

For example, a C₁₋₁₅ alkyl group such as n-butyl may be interrupted by an N(R) heterogroup as follows: —N(R)CH₂CH₂CH₂CH₃, —CH₂N(R)CH₂CH₂CH₃, —CH₂CH₂N(R)CH₂CH₃, or —CH₂CH₂CH₂N(R)CH₃. Similarly, a C₁₋₁₅ alkylene group such as n-butylene may be interrupted by an N(R) heterogroup as follows: —N(R)CH₂CH₂CH₂CH₂—, —CH₂N(R)CH₂CH₂CH₂—, —CH₂CH₂N(R)CH₂CH₂—, —CH₂CH₂CH₂N(R)CH₂— or —CH₂CH₂CH₂CH₂N(R)—. Typically, R is H or optionally substituted alkyl.

The term “hetero,” as used herein, pertains to compounds and/or groups which have at least one heteroatom, for example, multivalent heteroatoms (which are also suitable as ring heteroatoms) such as boron, silicon, nitrogen, phosphorus, oxygen, sulfur, and selenium (more commonly nitrogen, oxygen, and sulfur) and monovalent heteroatoms, such as fluorine, chlorine, bromine, and iodine.

“Optionally substituted”:

The phrase “optionally substituted”, as used herein, pertains to a group, as above, which may be unsubstituted or which may be substituted by one of the following substituent groups or one of the groups listed above:

Oxo (keto, -one): ═O.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₇ heterocyclyl group (also referred to as a C₃₋₇ heterocyclyloxy group), or a C₅₋₇ aryl group (also referred to as a C₅₋₇ aryloxy group), preferably a C₁₋₇ alkyl group.

C₁₋₇ alkoxy: —OR, wherein R is a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Thione (thioketone): ═S.

Imino (imine): ═NR, wherein R is an imino substituent, for example, hydrogen, C₁₋₇ alkyl group, a C₃₋₇ heterocyclyl group, or a C₅₋₇ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₇ heterocyclyl group (also referred to as C₃₋₇ heterocyclylacyl), or a C₅₋₇ aryl group (also referred to as C₅₋₇ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═O)NH(OH).

Ester (carboxylate, carboxylic acid ester, oxycarbonyl): —C(═O)OR, wherein R is an ester substituent, for example, a C₁₋₇ alkyl group, a C₃₋₇ heterocyclyl group, or a C₅₋₇ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₇ heterocyclyl group, or a C₅₋₇ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O) Ph, and —OC(═O)CH₂Ph.

Oxycarboyloxy: —OC(═O)OR, wherein R is an ester substituent, for example, a C₁₋₇ alkyl group, a C₃₋₇ heterocyclyl group, or a C₁₋₅₇ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR¹C(═O)R², wherein R¹ is an amide substituent, for example, hydrogen, a C₁₋₇ alkyl group, a C₃₋₇ heterocyclyl group, or a C₅₋₇ aryl group, preferably hydrogen or a C₁₋₇ alkyl group, and R² is an acyl substituent, for example, a C₁₋₇ alkyl group, a C₃₋₇ heterocyclyl group, or a C₅₋₇ aryl group, preferably hydrogen or a C₁₋₇ alkyl group. Examples of acylamide groups include, but are not limited to, —NHC(═O)CH₃, —NHC(═O)CH₂CH₃, and —NHC(═O) Ph. R¹ and R² may together form a cyclic structure, as in, for example, succinimidyl, maleimidyl, and phthalimidyl:

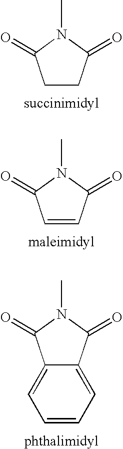

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₇ heterocyclyl group, or a C₅₋₇ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

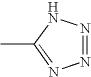

Amidine (amidino): —C(═NR)NR₂, wherein each R is an amidine substituent, for example, hydrogen, a C₁₋₇ alkyl group, a C₃₋₇ heterocyclyl group, or a C₅₋₇ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Cyano (nitrile, carbonitrile): —CN.

Isocyano: —NC.

Thiocyano (thiocyanato): —SCN.

Sulfhydryl (thiol, mercapto): —SH.

Thioether (sulfide): —SR, wherein R is a thioether substituent, for example, a C₁₋₇ alkyl group (also referred to as a C₁₋₇ alkylthio group), a C₃₋₇ heterocyclyl group, or a C₅₋₇ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₇ heterocyclyl group, or a C₅₋₇ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇ alkyl group, a C₃₋₇ heterocyclyl group, or a C₅₋₇ aryl group, preferably a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₇ heterocyclyl group, or a C₅₋₇ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₇ heterocyclyl group, or a C₅₋₇ aryl group, preferably a C₁₋₇alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₇ heterocyclyl group, or a C₅₋₇ aryl group, preferably a C₁₋₇alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₇ heterocyclyl group, or a C₅₋₇ aryl group, preferably a C₁₋₇ alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₇ heterocyclyl group, or a C₅₋₇ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₇ heterocyclyl group, or a C₅₋₇ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₇ heterocyclyl group, or a C₅₋₇ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇ alkyl group, a C₃₋₇ heterocyclyl group, or a C₅₋₇ aryl group, preferably a C₁₋₇ alkyl group. Examples of sulfinamino groups include, but are not limited to, —NHS(═O)CH₃ and —N(CH₃)S(═O)C₆H₅.

Phosphino (phosphine): —PR₂, wherein R is a phosphino substituent, for example, —H, a C₁₋₇alkyl group, a C₃₋₇heterocyclyl group, or a C₅₋₁₀aryl group, preferably —H, a C₁₋₇alkyl group, or a C₅₋₁₀aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)R₂, wherein R is a phosphinyl substituent, for example, a C₁₋₇alkyl group, a C₃₋₇heterocyclyl group, or a C₅₋₁₀aryl group, preferably a C₁₋₇alkyl group or a C₅₋₁₀aryl group. Examples of phosphinyl groups include, but are not limited to, —P(═O) (CH₃)₂, —P(═O) (CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O) (OR)₂, where R is a phosphonate substituent, for example, —H, a C₁₋₇alkyl group, a C₃₋₇heterocyclyl group, or a C₅₋₁₀aryl group, preferably —H, a C₁₋₇alkyl group, or a C₅₋₁₀aryl group. Examples of phosphonate groups include, but are not limited to, —P(═O) (OCH₃)₂, —P(═O) (OCH₂CH₃)₂, —P(═O) (O-t-Bu)₂, and —P(═O) (OPh)₂.

Phosphoric acid (phosphonooxy): —OP(═O)(OH)₂.

Phosphate (phosphonooxy ester): —OP(═O) (OR)₂, where R is a phosphate substituent, for example, —H, a C₁₋₇alkyl group, a C₃₋₇heterocyclyl group, or a CO₅₋₁₀aryl group, preferably —H, a C₁₋₇alkyl group, or a C₅₋₁₀aryl group. Examples of phosphate groups include, but are not limited to, —OP(═O) (OCH₃)₂, —OP(═O) (OCH₂CH₃)₂, —OP(═O) (O-t-Bu)₂, and —OP(═O) (OPh)₂.

Phosphorous acid: —OP(OH)₂.

Phosphite: —OP(OR)₂, where R is a phosphite substituent, for example, —H, a C₁₋₇alkyl group, a C₃₋₇heterocyclyl group, or a C₅₋₁₀aryl group, preferably —H, a C₁₋₇alkyl group, or a C₅₋₁₀aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR¹)—NR²₂, where R¹ and R² are phosphoramidite substituents, for example, —H, a (optionally substituted) C₁₋₇alkyl group, a C₃₋₇heterocyclyl group, or a C₅₋₁₀aryl group, preferably —H, a C₁₋₇alkyl group, or a C₅₋₁₀aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²₂, where R¹ and R² are phosphoramidate substituents, for example, —H, a (optionally substituted) C₁₋₇alkyl group, a C₃₋₇heterocyclyl group, or a C₅₋₁₀aryl group, preferably —H, a C₁₋₇alkyl group, or a C₅₋₁₀aryl group. Examples of phosphoramidate groups include, but are not limited to, —OP(═O) (OCH₂CH₃)—N(CH₃)₂, —OP(═O) (OCH₂CH₃)—N(i-Pr)₂, and —OP(═O) (OCH₂CH₂CN)—N(i-Pr)₂.

### Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms such as esters. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

### Quaternary Forms (—N⁺R¹R²R³, —N⁺R¹R²—, >N⁺R¹—) and Cationic Derivatives

The polymeric compounds of formulae I, III and IV described herein generally contain nitrogen atoms at various positions therein, including within terminal amino groups, e.g. R—NH₂; and within internal groups such as groups interrupting an alkyl or alkylene group within the polymer structure, e.g. R—N(H)—R′; and at the intersection of a polymer branch, e.g. R—N(—R′)—R″, wherein R, R′ and R″ may be alkylene groups as defined herein, for example.

In each case, reference to such a nitrogen atom, or to an amine or amino group containing such a nitrogen atom, includes the cationic derivative thereof. This includes derivatisation by protonation, e.g. by conversion of —NH₂, —NH—, or —N< to —N⁺H₃, —N⁺H₂— or —N⁺H< respectively; and by alkylation, e.g. by conversion of —NH₂, —NH—, or —N< to —N⁺RH₂, —N⁺RH—, >N⁺R— respectively, wherein R is an alkyl group as defined herein: preferably R is a methyl group. Thus, reference to such a nitrogen atom or amino or amine group includes the quaternary cationic derivative thereof. Thus, the compounds defined herein for use in the present invention include quaternary cationic derivatives thereof, which may include groups such as the terminal group —N⁺R¹R²R³, and the internal groups —N⁺R¹R²— (bidentate), and >N⁺R¹— (tridentate), wherein R¹, R² and R³ are preferably alkyl groups as defined herein. Various methods for synthesising quaternary cationic derivatives of nitrogen containing groups such as amine and amino groups are known to the skilled person, as described below and in WO 03/033027.

### Isomers, Salts, Solvates and Protected Forms

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

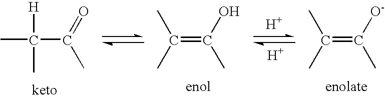

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

A wide variety of such “protecting”, “blocking”, or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇ alkyl ester (e.g., a methyl ester; a t-butyl ester); a C₁₋₇ haloalkyl ester (e.g., a C₁₋₇trihaloalkyl ester); a triC₁₋₇ alkylsilyl-C₁₋₇ alkyl ester; or a C₅₋₇ aryl-C₁₋₇ alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. Suitable dose ranges will typically be in the range of from 0.01 to 20 mg/kg/day, preferably from 0.1 to 10 mg/kg/day.

Compositions and their Administration

Compositions (e.g. pharmaceutical compositions) may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, transdermal, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium, carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

### Acronyms

For convenience, many chemical moieties are represented using well known abbreviations, including but not limited to, methyl (Me), ethyl (Et), n-propyl (nPr), iso-propyl (iPr), n-butyl (nBu), sec-butyl (sBu), iso-butyl (iBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), acetonitrile (ACN), trifluoroacetic acid (TFA), dimethylformamide (DMF), tetrahydrofuran (THF), and dimethylsulfoxide (DMSO).

### Polyethylenimine (PEI)

The compound of formula I of the first and third aspects of the invention may be a polyethylenimine compound.

Polyethylenimine (PEI) is an aliphatic polyamine characterized by the repeating chemical unit denoted as —(CH₂—CH₂—NH)—.

PEI may be branched or linear. Preferably, the PEI used in the present invention is linear PEI. However, the use of branched PEI is also envisaged.

The amine groups of PEI exist in primary, secondary and tertiary form. In its branched form, primary, secondary and tertiary amine groups exist in the approximate ratio of 1:2:1 with a branching site every 3-3.5 nitrogen atoms along any given chain segment. The primary amine groups are chain-terminating units, and are the most basic and chemically reactive. Branched PEI is commercially available. For example, branched PEI having a molecular weight of 25 kDa is available from Aldrich, and is described in Cancer Gene Therapy (2002) 9, 673-680.

However, PEI with fewer branching sites is also known, and linear PEI is described in J. Controlled Release 91 (2003) 201-208, and in Cancer Gene Therapy (2002) 9, 673-680. Linear PEI having a molecular weight of 22 kD is commercially available from Helena Biosciences, UK, and St. Leon-Rot, Germany.

PEI has a wide molecular weight range, for example, PEI molecular weights ranging from 300 daltons to 800 kD are known.

Additionally, PEI is a cationic polymer, characterized by a high charge density at neutral pH (pH 7). For example, the cationic charge density of PEI may be in excess of 20 meq/g. Thus, PEI is positively charged at physiological pH (generally considered to be 7.4).

As the molecular weight of PEI increases, the polymer structure is believed to assume a characteristic spherical configuration. This implies that there are charged nitrogen groups both on the surface and in the sterically protected interior of the molecule. PEIs are produced commercially as viscous liquids, both in the anhydrous and aqueous solution form. The viscosity of PEI is directly proportional to its concentration and molecular weight. PEIs are infinitely soluble in most polar materials including water, alcohols, glycols and certain organic solvents. Anhydrous PEIs will generate considerable heat upon aqueous dissolution due to an exothermic heat of dilution.

The most prominent feature of PEI is its extremely high cationic charge density. The repeating monomer unit contains one protonatable nitrogen atom for every unit weight of 42. By theory, supported in practice by titrimetric analytical measurements, PEI has the highest cationic charge density (20-25 milliequivalents per gram) of any known organic polymer. Since PEI does not normally contain an appreciable amount of quaternary groups, it achieves its cationicity through protonation of the amine groups from the surrounding medium. This leads to a correlation between pH and cationic charge density. However, adhesive strength is not often affected in non-protonated environments because hydrogen bonding and Van der Waal's forces also participate in the bonding mechanism.

PEI may be derivatised to contain cationic quaternary ammonium groups. For example, the terminal amino groups of PEI may be converted to a quaternary form in which three alkyl groups as defined herein are covalently bound to the nitrogen atom of the terminal amino group. Preferably, substantially only the terminal (primary) amino groups are converted to the quaternary form. However, in other embodiments, conversion of amino groups other than the terminal amino groups, i.e. internal (secondary and tertiary) amino groups, to the corresponding quaternary forms is also envisaged.

### Dendrimers

The compounds of formula III of the second aspect of the invention are dendrimer compounds.

Dendrimer synthesis is a field of polymer chemistry defined by regular, highly branched monomers leading to a monodisperse, tree-like or generational structure.

Synthesizing monodisperse polymers demands a high level of synthetic control which is achieved through stepwise reactions, building the dendrimer up one monomer layer, or “generation,” at a time. Thus, each dendrimer used in the present invention, consists of a multifunctional core molecule with a dendritic wedge attached to each functional site of the core. The functional sites of the core may be amino groups, for example. Preferably, each of the dendritic wedges is covalently bonded to a core functional atom of the functional site of the core. If the core functional sites are amino groups, then the core functional atoms are the nitrogen atoms of the amino groups, and each dendritic wedge is bonded to a nitrogen atom of the core. Similarly, if the core functional sites are phosphine groups, phosphate groups or other phosphorus-containing functional groups (e.g. derived from one of the phosphorus-containing substituents defined above), then the core functional atoms could be the phosphorus atoms of the phosphorus-containing groups, and each dendritic wedge would be bonded to a phosphorus atom of the core. Of course, cores containing other types of functional atoms may also be used in the dendrimers employed in the present invention, such as cores with C, S or O functional atoms, or wherein the functional atoms are other heteroatoms. The core molecule is referred to as “generation 0.” Each successive repeat unit along all branches forms the next generation, “generation 1,” “generation 2,” and so on until the nth terminating generation.

There are two defined methods of dendrimer synthesis, divergent and convergent. In the divergent method the molecule is assembled from the core to the periphery; while in the convergent method, the dendrimer is synthesized beginning from the outside and terminating at the core. Generally, in either method the synthesis requires a stepwise process, attaching one generation to the last, purifying, and attaching the next generation.

### Diaminobutane (DAB) Polypropylenimine (PPI) Dendrimers

The compounds of formula III of the second aspect of the invention may be polypropylenimine (PPI) dendrimer compounds based on the polypropylenimine repeat unit —(CH₂—CH₂—CH₂—N)<, wherein the N atoms of the repeat units of a given generation are covalently bonded to two repeat units of the next generation, as follows:

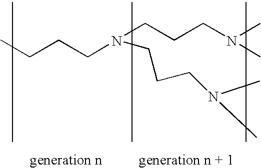

Many commercially available PPI dendrimers are based on a 1,4-diaminobutane core, and are thus referred to as “DAB” dendrimers. Such PPI DAB dendrimers are described in the published PCT application WO 03/033027, and in Pharmaceutical Research (2004) Vol. 21, No. 3, 458-466. Such dendrimers are commercially available from Aldrich (Poole, UK): see http://www.sigmaaldrich.com/img/assets/12141/Dendrimers_macro32_{—}14.pdf

Such DAB dendrimers are referred to as DAB prefixed to the number of surface amine groups. Thus, DAB 4, is a generation 1 dendrimer with four —CH₂—CH₂—CH₂—NH₂ units covalently bonded to the two nitrogen atoms of the 1,4-diaminobutane core, as follows:

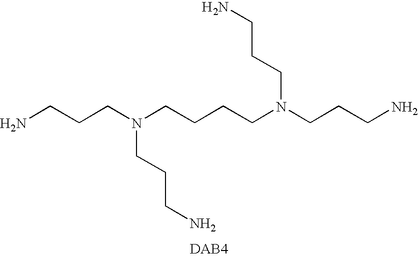

Similarly, DAB 8 is a generation 2 dendrimer with eight —CH₂—CH₂—CH₂—NH₂ units covalently bonded to the four terminal nitrogen atoms of DAB 4, as follows:

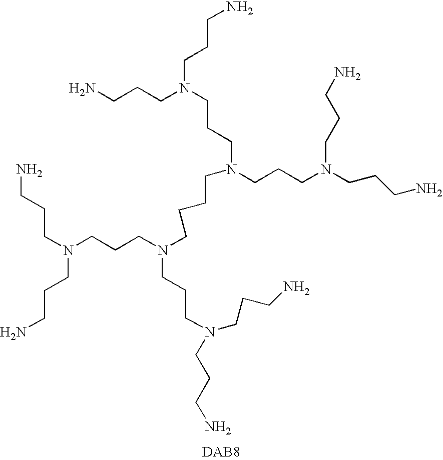

Similarly, DAB 16 is a generation 3 dendrimer with sixteen —CH₂—CH₂—CH₂—NH₂ units covalently bonded to the eight terminal nitrogen atoms of DAB 8, as follows:

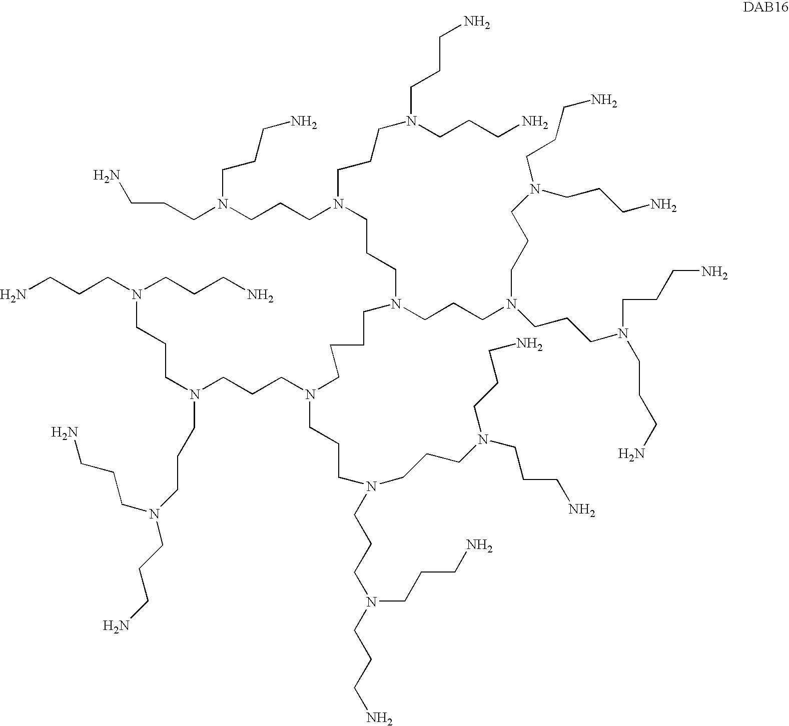

Similarly, DAB 32 is a generation 4 dendrimer with 32-CH₂—CH₂—CH₂—NH₂ units covalently bonded to the sixteen terminal nitrogen atoms of DAB 16, and DAB 64 is a generation 5 dendrimer with 64-CH₂—CH₂—CH₂—NH₂ units covalently bonded to the 32 terminal nitrogen atoms of DAB 32.

Polypropylenimine (PPI) dendrimers contain protonatable nitrogens in the form of amine groups (both surface primary amino groups and internal amine groups). Thus, the PPI dendrimers used in the present invention, such as the “DAB” dendrimers described above, are cationic, and have an overall cationic (positive) charge at neutral pH (pH 7). Thus, the PPI dendrimers used in the present invention are positively charged at physiological pHs of around 7 (e.g. 7.4). These dendrimers do not normally contain an appreciable amount of quaternary groups. Thus, they achieve their cationicity through protonation of the amine groups from the surrounding medium. This leads to a correlation between pH and cationic charge density.

However, PPI dendrimers such as the commercially available DAB dendrimers DAB4, DAB8, DAB16, DAB32 and DAB64 may be quaternised (as described below, under “synthesis of quaternised DABs”). Thus, PPI dendrimers may be derivatised to contain cationic quaternary ammonium groups.

It is preferable that the terminal amino groups (e.g. —NRR′, where R and R′ are independently H or alkyl as defined herein) of the PPI dendrimers are converted to a quaternary form in which three alkyl groups as defined herein are covalently bound to the nitrogen atom of the terminal amino group. Preferably, these alkyl groups are methyl groups. Preferably, substantially only the terminal amino groups are converted to the quaternary form. However, in other embodiments, conversion of non-terminal (internal) amino groups to the corresponding quaternary form is envisaged.

DAB dendrimers, such as DAB4, DAB8, DAB16, DAB32 and DAB64 may be quaternarised such that the terminal amino groups are converted to the quaternary form. An example is QDAB16, which is described in WO 03/033027 and has the following structure:

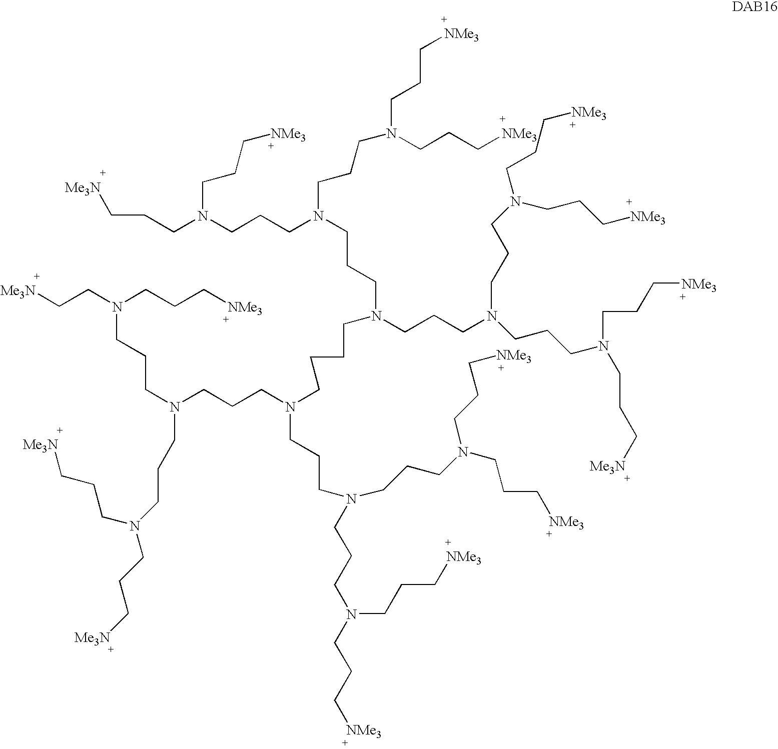

QDAB4, QDAB8, QDAB16, QDAB32 and QDAB64 have analogous structures. It is particularly preferred that DAB8 is used in the present invention in the quaternary form, thus QDAB8 is more preferable than DAB8. This is because quaternised DAB8 has a lower in vivo toxicity than non-quaternised DAB8.

The synthesis and structure of DAB PPI dendrimers is further described in WO 03/033027.

### Polyamidoamine (PAMAM) Dendrimers

The compounds of formula III of the second aspect of the invention may be PAMAM dendrimer compounds based on the amidoamine repeat unit —(CH₂—CH₂—C(═O)—N(H)—CH₂—CH₂—N)<, wherein the amine N atoms (as opposed to the amido N atoms) of the repeat units of a given generation are covalently bonded to two repeat units of the next generation, as follows:

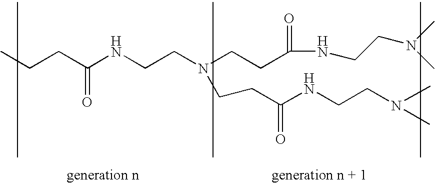

PAMAM dendrimers are commercially available (e.g. from Sigma-Aldrich), and core structures of these dendrimers include ethylenediamine, 1,4-diaminobutane, 1,6-diaminohexane, 1,12-diaminododecane. For details of commercially available PAMAM dendrimers, see: http://www.sigmaaldrich.com/img/assets/12141/Dendrimers_macro32_{—}14.pdf and http://www.sigmaaldrich.com/Area_of_Interest/Chemistry/Materials_Science/Nanomaterials/Dendrimers.html

A generation 0 PAMAM dendrimer with a core structure based on ethylene diamine is shown below:

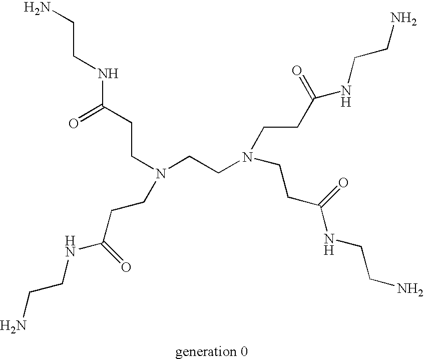

An example of a generation 1 PAMAM dendrimer is when eight —(CH₂—CH₂—C(═O)—N(H)—CH₂—CH₂—N)< units are covalently bonded to the four terminal nitrogen atoms of the generation 0 dendrimer shown above. Similarly, a generation 2 PAMAM dendrimer with a core structure based on ethylenediamine is shown below, in which sixteen amidoamine units are bonded to the eight terminal nitrogen atoms of the generation 1 dendrimer described above:

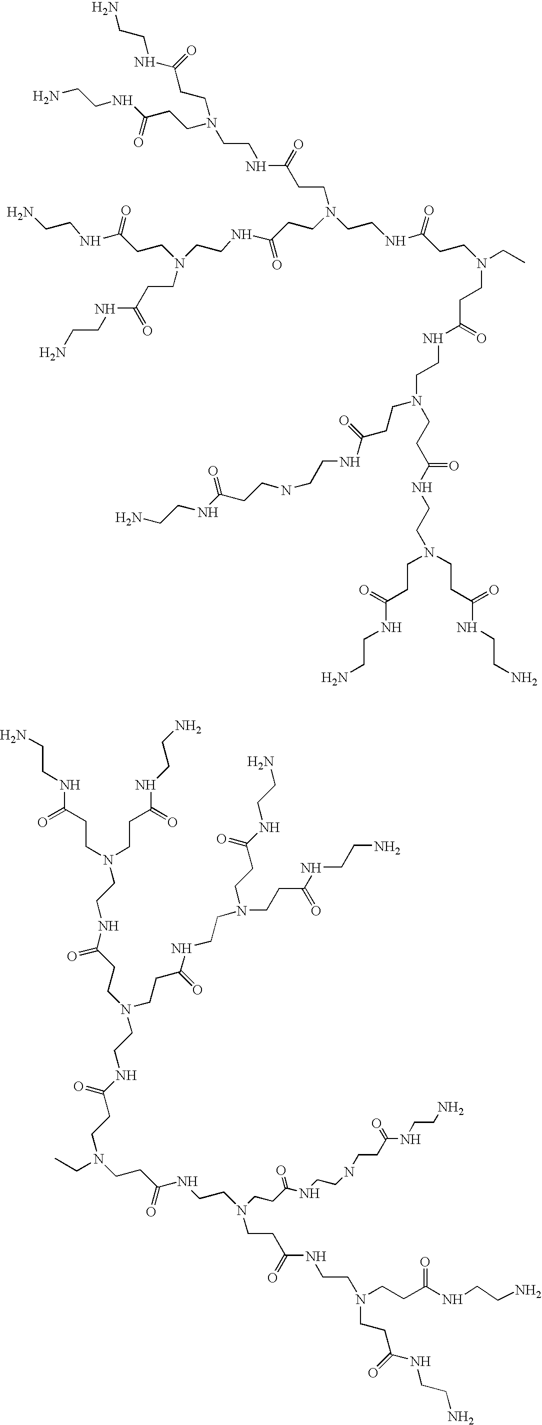

PAMAM dendrimers having generation numbers in the range 0 to 10 are commercially available from Sigma-Aldrich.

PAMAM dendrimers may be based on a variety of different core molecules. These include diaminoalkane molecules such as ethylenediamine and 1,4-diaminobutane which both yield dendrimers with 4-fold core geometry. However, core molecules can also be (or be derived from) ammonia or tris(2-aminoethyl)amine (TAEA), which yield dendrimers with a 3-fold core geometry. The synthesis of PAMAM dendrimers based on a variety of different core geometries is described in Bioconjugate Chem. (1996) 7, 703-714.

The PAMAM dendrimers used in the present invention are cationic, and have an overall cationic (positive) charge at neutral pH (pH 7). Thus, the PAMAM dendrimers used in the present invention are positively charged at physiological pH (e.g. 7.4). These dendrimers do not normally contain an appreciable amount of quaternary groups. Thus, they achieve their cationicity through protonation of the amine groups from the surrounding medium. This leads to a correlation between pH and cationic charge density.

However, the terminal amino groups of the PAMAM dendrimers may be converted to a quaternary form in which three alkyl groups as defined herein are covalently bound to the nitrogen atom of each terminal amino group. Preferably, these alkyl groups are methyl groups. Preferably, substantially only the terminal amino groups are convered to the quaternary form. However, in other embodiments, conversion of non-terminal (internal) amino groups to the corresponding quaternary forms is envisaged.

PAMAM dendrimers may be derivatised with surface groups such as optionally substituted C₁₋₁₆ alkyl groups as defined herein, which are optionally interrupted with one or more heteroatoms or heterogroups, including other forms such as salts or derivatives thereof. Examples of such groups include amidoethylethanolamine, hexylamide, succinamic acid, Tris(hydroxymethyl)amidomethane, amidoethanol, amino and carboxylate (e.g. sodium carboxylate) groups. PAMAM dendrimers with these exemplified surface groups are available from Sigma-Aldrich.

A further example of a PAMAM dendrimer compound for use in the present invention is SuperFect, which is an activated, spherical PAMAM dendrimer that possesses radiating branches with charged terminal amino groups, and is commercially available from Quiagen. See: http://www1.qiagen.com/Products/Transfection/TransfectionReagents/SuperFectTransfectionReagent.aspx See also the SuperFect transfection reagent handbook at: http://www1.qiagen.com/literature/handbooks/PDF/Transfection/TF_S uperFect/1023348_HB_SF_{—}1202.pdf

See also Tang, M. X. and F. C. Szoka (1997). “The influence of polymer structure on the interactions of cationic polymers with DNA and morphology of the resulting complexes.” Gene Therapy 4(8): 823-832; and U.S. Pat. No. 5,990,089 “Self-assembling polynucleotide delivery system comprising dendrimer polycations”.

Reference to the dendrimer compounds of formula III, for use in the second aspect of the invention (as active agents in the preparation of a medicament for the treatment of a condition characterised by undesirable cellular proliferation), includes activated or fractured (e.g. heat fractured) derivatives thereof, including activated SuperFect or fractured SuperFect, which is commercially available from Quiagen.

Dendrimers for use in the present invention can be modified by covalently binding derivatising groups, such as hydrophobic or hydrophilic groups, or a combination of hydrophobic and hydrophilic substitutions to make the dendrimers amphiphilic. Such groups may be attached to the surface of a dendrimer. Additionally, two dendrimer molecules may be attached to either end of a hydrocarbon chain with a carbon length of 8, 12, 14, 16 or 18 carbon atoms to give bolamphiphilic dendrimers. The number of derivatising groups may vary from one derivatising group per dendrimer molecule up to and including derivatising all available surface or terminal groups on the dendrimer molecule, for example, derivatising all 8 surface groups of the DAB8 molecule or all 16 surface groups of the DAB16 molecule. An example of a preferred derivatising group is hyaluronic acid. Derivatising dendrimer molecules is described in WO 03/033027.

### General Synthesis Methods

Methods for the chemical synthesis of compounds for use in the present invention are described herein. These methods may be modified and/or adapted in known ways in order to facilitate the synthesis of additional compounds within the scope of the present invention. Descriptions of general laboratory methods and procedures, useful for the preparation of the compounds of the present invention, are described in Vogel's Textbook of Practical Organic Chemistry (5^{th} edition, Ed. Furniss, B. S., Hannaford, A. J., Smith, P. W. G., Tatchell, A. R., Longmann, UK).

In the methods described below, other substituent groups to those introduced may be present as precursors of those groups, or as protected versions of those groups.

Dendrimer compounds of formula III can be prepared in a stepwise fashion from simple monomer units, the nature and functionality of which can be easily controlled and varied. Dendrimers are synthesised by the repeated addition of building blocks to a multifunctional core (divergent approach to synthesis) or towards a multifunctional core (convergent approach to synthesis), and each addition of a 3-dimensional shell of building blocks leads to the formation of a higher generation of the dendrimers. See Bosman, A. W. et al. (1999) “About dendrimers: structure, physical properties, and applications” Chem. Rev. 99, 1665-1688.

Polypropylenimine dendrimers may start from a diaminoalkane core (e.g. 1,4-diaminobutane) to which is added twice the number of amino groups by a Michael addition of acrylonitrile to the primary amines followed by the hydrogenation of the nitriles. This results in a doubling of the amino groups. See De Brabander-van den Berg, E. M. M. et al. (1993) “Poly(propylene imine) dendrimers: large scale synthesis by heterogeneously catalysed hydrogenations” Angew. Chem. Int. Ed. Engl. 32, 1308-1311.

The synthesis of PAMAM dendrimers involves the stepwise, exhaustive addition of two monomers, methacrylate and ethylenediamine. Two methacrylate monomers add to each bifunctional ethylenediamine, leading to increasingly branched structures with each cycle or generation. Scheme 1 below shows the stepwise addition of methacrylate and ethylenediamine to ammonia, tris-(2-aminoethyl)amine and ethylenediamine cores (each of which are examples of core molecules) to synthesis PAMAM dendrimers having three- and four-fold core geometries. The synthesis of dendrimers according to this principle is described in Bioconjugate Chem. (1996) 7, 703-714 and by Tomalia, D. A. et al. “A new class of polymers: Starburst-dendritic macromolecules” Polymer J. (1985) 17, 117-132 and Tomalia, D. A. et al. (1990) “Starburst dendrimers: Molecular-level control of size, shape, surface chemistry, topology, and flexibility from atoms to macroscopic matter” Angew. Chem. Int. Ed. Engl. 29, 138-175.

### Scheme 1

### PAMAM Dendrimer Synthesis

(i) Ammonia-based core (3-fold core geometry)

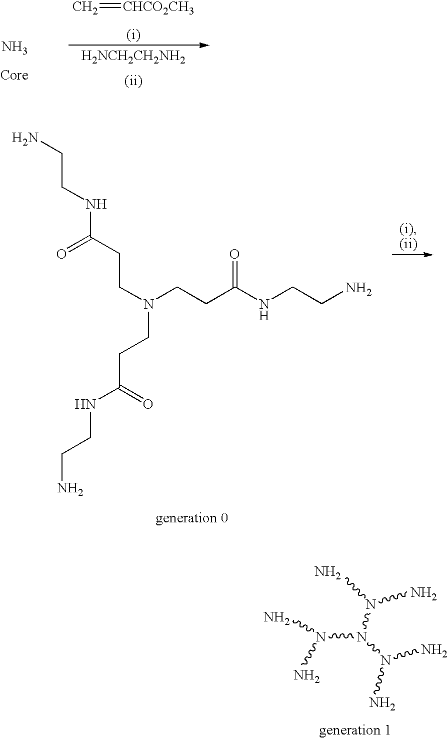

(ii) Tris(2-aminoethyl)amine-based core (3-fold core geometry)

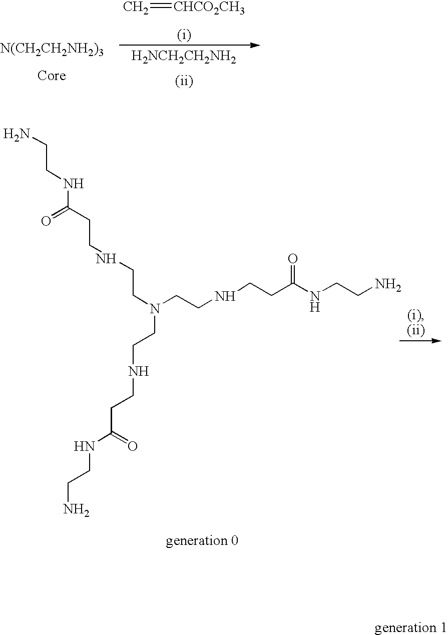

(iii) Ethylenediamine-based core (4-fold core geometry)

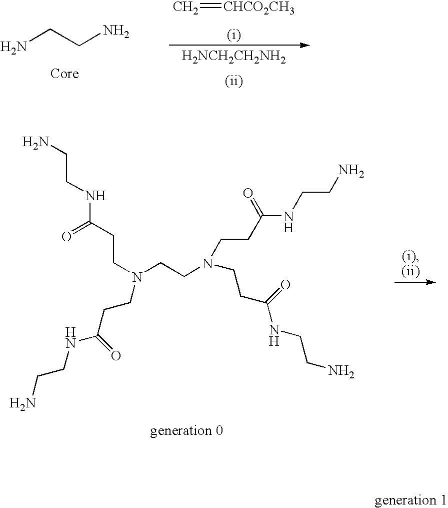

Certain compounds for use in the present invention, such as polyethylenimine polymers (PEIs), and the PPI and PAMAM dendrimers (including SuperFect), are commercially available or can be derived from such compounds. PEIs are produced commercially as viscous liquids, both in the anhydrous and aqueous solution form.

### Preferences

The following preferences may be combined with one another, and may be different for each aspect of the present invention.

Preferably, in formula III of the second aspect of the invention, the C₁₋₁₆ alkyl and C₁₋₁₆ alkylene groups are optionally substituted by one or more groups selected from oxo, amino, hydroxy, carboxy, alkoxy, ester and halo.

Preferably, neither X nor X₂ nor X₃ of a given generation of the dendrimer is N(R²) when Y of that generation is N. N(R²) is as defined above in the second aspect of the invention.

Preferably, when Y of a given generation of the dendrimer is C(R¹), X of that generation is selected from N(R²) and optionally substituted C₁₋₁₆ alkylene interrupted by one or more N(R²) groups. Additionally or alternatively, when Y of a given generation of the dendrimer is C(R¹), both X₂ and X₃ of that generation are independently selected from N(R²) and optionally substituted C₁₋₁₆ alkylene interrupted by one or more N(R²) groups.

Preferably the generation number, n, of the dendrimer is in the range 1 to 10. More preferably, the generation number, n, is in the range 1 to 6.

It is preferred that Y is N in one or more of the generations of the dendrimer. For example, if n is 4, is preferred that Y is N in at least one of the generations of the dendrimer. It is more preferred that Y is N in at least 2 of the generations of the dendrimer. It is even more preferred that Y is N in at least three of the generations of the dendrimer. It is most preferred that Y is N in all four of the generations of the dendrimer. This preference applies to other values of n: it is least preferred that Y is N in none of the generations, it is more preferred that Y is N in at least one of the generations, and so-on, until it is most preferred that Y is N in all of the generations.

Thus, preferably, Y is N in at least 50% of the generations of the dendrimer: it is preferred that in most of the generations, the dendrimer branches at nitrogen atoms rather than carbon atoms.

Additionally or alternatively, it may be that in at least 50% of the generations of the dendrimer, X is selected independently for each of said generations of the dendrimer from N(R²) and optionally substituted C₁₋₁₆ alkylene interrupted by one or more N(R²) groups. Thus, in this arrangement, most of the generations contain a nitrogen atom, even though Y may not be N in any, some or all of the generations. Additionally or alternatively, it may be that in at least 50% of the generations of the dendrimer, X₂ and X₃ are independently selected, independently for each of said generations of the dendrimer, from N(R²) and optionally substituted C₁₋₁₆ alkylene interrupted by one or more N(R²) groups. Again, in this arrangement, most of the generations contain a nitrogen atom, even though Y may not be N in any/some/all of the generations.

Preferably, in at least 50% of the generations of the dendrimer, Y is N, X₂ and X₃ are single bonds, and X is selected from optionally substituted C₁₋₁₆ alkylene groups independently for each of said at least 50% of the generations of the dendrimer, wherein said C₁₋₁₆ alkylene groups are independently optionally interrupted by one or more N(R²) or O heterogroups.

Preferably, T₁ and T₂ are independently selected from H, hydroxy, carboxy, halo and optionally substituted amino, amido, alkoxy, acyl, ester, C₁₋₁₆ alkyl, C₃₋₇ heterocyclyl, C₅₋₁₀ aryl, C₅₋₁₀ heteroaryl, C₁₋₁₆ alkylene-NR³R⁴, C₅₋₁₀ arylene-NR³R⁴, C₁₋₁₆ alkylene-C₅₋₁₀ arylene-NR³R⁴, and C₅₋₁₀ arylene-C₁₋₁₆ alkylene-NR³R⁴, wherein R³ and R⁴ are independently selected from H and optionally substituted C₁₋₁₆ alkyl and C₅₋₁₀ aryl, wherein said C₁₋₁₆ alkyl and C₁₋₁₆ alkylene groups are optionally interrupted by one or more N(R²) or O heterogroups. More preferably, T₁ and T2 are independently selected from H, C₁₋₁₆ alkyl and C₁₋₁₆ alkylene-NR³R⁴, wherein R³ and R⁴ are independently selected from H and optionally substituted C₁₋₁₆ alkyl, wherein said C₁₋₁₆ alkyl and C₁₋₁₆ alkylene groups are optionally interrupted by one or more N(R²) or O heterogroups.

Preferably Y of the nth generation is N, and X₂ and X₃ of the nth generation are single bonds, so that the dendrimer has terminal groups NT₁T₂. Here, the “nth generation” means the final generation of the dendrimer, to which the end groups T₁ and T₂ are bonded.

Preferably, the dendrimer has an overall cationic charge (i.e. it is positively charged overall) at physiological pH (e.g. pH 7.4).

Preferably this overall cationic charge arises as a result of the dendrimer containing nitrogen atoms at various positions therein, including within terminal amino groups, e.g. L-NH₂ or L-NR′₂ and/or within internal groups (denoted “internal nitrogen-containing groups”) such as groups interrupting an alkyl or alkylene group within a linear part of the polymer structure, e.g. L-N(H)-L′ or L-N(R′)-L′; or at the intersection of a polymer branch, e.g. L-N(-L′)-L″, wherein L, L′ and L″ may be alkylene groups as defined herein, and R¹ may be an alkyl group as defined herein, for example.

The terminal amino groups and/or internal nitrogen-containing groups preferably have pKa's which cause them to be protonated, and therefore cationic, at physiological pH. Preferably, terminal amino groups and/or internal nitrogen-containing groups of the dendrimer have pKa's above 7, more preferably above 7.5, and most preferably in the range 8 to 12.

However, it may be that only terminal amino groups of the dendrimer (and not internal nitrogen-containing groups) have such preferable pKa values. Indeed, the pKa values of terminal amino groups would generally be expected to be within this preferred pKa range, and hence protonated and cationic at physiological pH. This is exemplified by the following pKa values (all in the range 9-11), which correspond to the pKa's of the α-NH₃⁺ groups of the following amino acids (see Stryer, L.; “Biochemistry”; Third Edition; W.H. Freeman and Company, New York; page 42; ISBN 0-7167-1920-7): Alanine, 9.9; Glycine, 9.8; Phenylalanine, 9.1; Serine, 9.1; Valine, 9.6; Aspartic acid, 10.0; Glutamic acid, 9.7; Histidine, 9.2; Cysteine, 10.8; Tyrosine, 9.1; Lysine, 9.2; and Arginine, 9.0.

Thus, it is preferred that the terminal groups or “surface groups” of the dendrimer (that is, groups that are bonded to or part of the final, nth generation of the dendrimer, or that are bonded to or part of the T₁ and T₂ groups) are predominantly cationic at physiological pH. Preferably these groups have pKa's above 7, more preferably above 7.5, and most preferably in the range 8 to 12. Preferably, these terminal groups include amino groups, which are cationic at physiological pH.

Preferably, the terminal groups of the dendrimer are not carboxyl groups, or do not comprise carboxyl groups, because carboxyl groups are generally anionic at physiological pH. Similarly, it is preferred that the terminal groups of the dendrimer do not comprise sulphonic acid groups, or naphthyl 3,6-disulphonic acid groups, or salts thereof.

Although dendrimer compounds having carboxyl, sulphonic acid, or naphthyl 3,6-disulphonic acid substituents are envisaged, it is preferable that the dendrimer retains a predominantly cationic charge (an overall positive charge) at physiological pH. Thus, it is preferred that the dendrimer compounds described herein are not predominantly anionic (that is, they should not be negatively charged overall) at physiological pH. They carry more positive charges than negative charges at physiological pH.

Preferably, X₂ and X₃ are single bonds and Y is N so that the dendrimer compound is of the general formula IV:

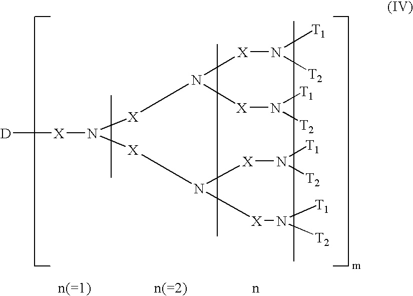

wherein
m is an integer from 2 to 8;
X is selected from C₁₋₁₆alkylene groups independently for each generation of the dendrimer;
wherein each of said C₁₋₁₆ alkylene groups is optionally interrupted by one or more N(R²) or O heterogroups and optionally substituted by one or more groups selected from oxo, amino, hydroxy, carboxy, alkoxy, ester and halo.

Preferably, said functional atoms of the core are selected from nitrogen, phosphorus, oxygen, carbon or sulphur. More preferably each of said functional atoms of the core (to which the X groups of the first generation are bonded) is nitrogen.

Preferably, D is a hydrocarbon, such as a saturated or unsaturated aliphatic or alicyclic hydrocarbon or an aromatic hydrocarbon, (or a combination of said different types of hydrocarbons bonded to each other) wherein the hydrocarbon is optionally substituted, and optionally interrupted by one or more heteroatoms. Preferably said hydrocarbon has from 1 to 16 carbon atoms. Preferably said hydrocarbon comprises one or more substituent groups, selected or derived from the substituent groups defined herein. Preferably, each substituent group comprises a core functional atom that is bonded to one or more X groups of the first generation of the dendrimer. Preferably each core functional atom is bonded to one or two X groups of the first generation of the dendrimer. Preferably, the number of substituent groups is 2, 3 or 4, each comprising a core functional atom bonded to one or more (preferably one or two) X groups of the first generation of the dendrimer. Additionally or alternatively, the hydrocarbon itself may comprise core functional atoms, e.g. carbon core functional atoms that are part of the hydrocarbon structure and additionally bonded to one or more (preferably one or two) X groups of the first generation of the dendrimer, or heteroatoms by which the hydrocarbon structure is interrupted and which are additionally bonded to one or more (preferably one or two) X groups of the first generation of the dendrimer.

While it is preferable that D is an organic core molecule, as described above, inorganic core molecules are also envisaged. An example of an inorganic core is an alternating nitrogen-phosphorus heterocyclic ring structure, having phosphorus and/or nitrogen core functional atoms bonded to X groups of the first generation of the dendrimer.

Preferably, D is selected from the following core structures, in which the core functional atom is nitrogen:

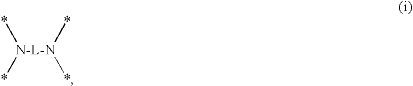

wherein m is 4 and L is C₁₋₁₆ alkylene;

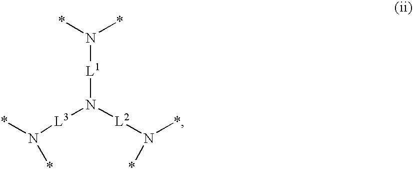

wherein m is 6 and L¹, L² and L³ are independently selected from C₁₋₁₆ alkylene groups;

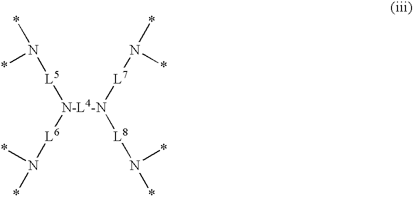

wherein m is 8 and L⁴, L⁵, L⁶, L⁷ and L⁸ are independently selected from C₁₋₁₆ alkylene groups; and

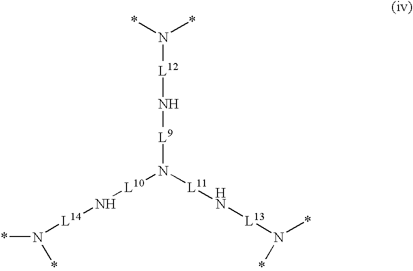

wherein m is 6; L⁹, L¹⁰ and L¹¹ are independently selected from C₁₋₄ alkyl groups; and L¹, L¹³ and L¹⁴ are independently selected from C₁₋₁₆ alkylene groups;
wherein * represents a point of covalent attachment to an X group of the first generation, and wherein each of said C₁₋₁₆ alkylene groups is optionally interrupted by one or more N(R²) or O heterogroups and optionally substituted by one or more groups selected from oxo, amino, hydroxy, carboxy, alkoxy, ester and halo.

Preferably m is an integer from 4 to 8. Most preferably, m is 4 or 8.

L, L¹, L², L³, L⁴, L⁵, L⁶, L⁷, L⁸, L¹², L¹³ and L¹⁴ may be independently selected from linear, unsubstituted C₁₋₁₂ alkylene groups, and L⁹, L¹⁰, L¹¹ are independently selected from linear, unsubstituted C₁₋₄ alkyl groups.

For example, when D is

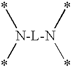

L may be ethylene, propylene, butylene, hexylene or dodecylene. Preferably, L is butylene.

Alternatively, D may be

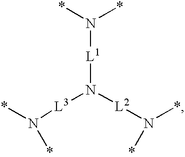

wherein L¹, L², and L³ may be selected from groups having the general structure Cₚ alkylene-C(O)N(R²)—C_{q} alkylene wherein p and q are integers and p+q is in the range 2 to 16. Preferably, each of L¹, L² and L³ is —(CH₂)₂—C(═O)N(H)—(CH₂)₂—, for example in a PAMAM dendrimer.

Alternatively, D may be

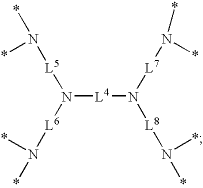

wherein L⁴ is a linear unsubstituted C₁₋₁₂ alkylene group. L⁵, L⁶, L⁷ and L⁸ may be selected from groups having the general structure Cₚ alkylene-C(O)N(R²)—C_{q} alkylene wherein p and q are integers and p+q is in the range 2 to 16. Preferably, each of L⁵, L⁶, L⁷ and L⁸ is —(CH₂)₂—C(═O)N(H)—(CH₂)₂—. L⁴ is preferably ethylene, propylene, butylene, hexylene or dodecylene. More preferably, L₄ is ethylene, for example in a PAMAM dendrimer, or butylene, for example in a poly(propylenimine) (PPI) dendrimer.

Alternatively, D is

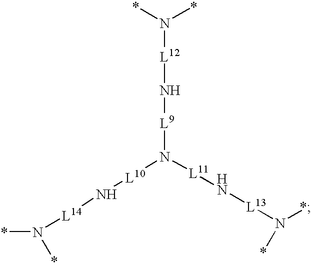

wherein L⁹, L¹⁰ and L¹¹ are linear unsubstituted C₁₋₄ alkylene groups. Preferably, L¹², L¹³ and L¹⁴ are selected from groups having the general structure Cₚ alkylene-C(O)N(R²)—C_{q} alkylene wherein p and q are integers and p+q is in the range 2 to 16. Preferably, each of L¹², L¹³ and L¹⁴ is —(CH₂)₂—C(═O)N(H)—(CH₂)₂—, for example in a PAMAM dendrimer. Preferably, each of L⁹, L¹⁰ and L¹¹ is ethylene.

Alternatively, D is

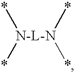

wherein m is 4 and L is selected from C₅₋₁₀ arylene, C₁₋₁₅ alkylene-C₅₋₁₀ arylene, C₁₋₁₅ alkylene-C₅₋₁₀ arylene-C₁₋₁₅ alkylene-, or C₅₋₁₀ arylene-C₁₋₁₅ alkylene-C₅₋₁₀ arylene.

Alternatively, D is a substituted C₅₋₁₀ aryl group, wherein the substituents comprise the core functional atoms (e.g. nitrogen atoms). For example, D may be

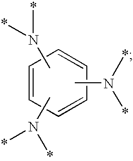

a trisubstituted phenyl ring, wherein m is 6 and the three-substituents are either bonded respectively to the 1, 2, and 3 positions; the 1, 2 and 4 positions; or the 1, 3 and 5 positions of the phenyl ring. The phenyl ring may be optionally substituted at the other positions, with a substituent as defined herein.

In the above D groups, each nitrogen atom is bonded to two X groups of the first generation: accordingly, m is twice the number of core functional nitrogen atoms in each case. However, other core structures are envisaged, similar to those listed above, but wherein one or more of the core functional nitrogen atoms are (each) only bonded to one X group of the first generation of the dendrimer, rather than two X groups. Accordingly, in these alternative D groups m is less than twice the number number of core functional nitrogen atoms. In these alternative D groups, the nitrogen atoms not bonded to two X groups may be bonded instead to one X group and one substituent as defined herein (e.g. H or alkyl).

While nitrogen core functional atoms are preferred, cores having other functional atoms bonded to the X groups of the first generation of the dendrimer are also envisaged. These core functional atoms may be heteroatoms such as phosphorus, sulphur, and oxygen; or carbon, for example. A combination of different types of core functional atoms may be employed in a single core structure, although it is preferable that the core functional atoms within a given core structure are the same type (e.g. all nitrogen, or all phosphorus).

A phosphorus core functional atom may be part of a phosphine, phosphine oxide or phosphate group (or another group derived from one of the phosphorus-containing functional groups defined herein) which is bonded to or part of the core structure. For example, core structures similar to those listed above are envisaged, in which the terminal nitrogen atoms (the core functional atoms) are replaced with trivalent phosphorus atoms (—P<), or pentavalent phosphine oxide groups (—P(═O)<). Phosphorus-containing core structures are known in the art, and may be employed in the present invention. See http://www.dendrichem.com/uk/17.htm for examples of phosphorus-containing core structures.

Similarly, a carbon core functional atom may be part of a carbonyl group, for example (or part of another group derived from one of the carbon-containing functional groups defined herein, including alkyl and aryl groups) which group is bonded to or part of the core structure. For example, core structures D having one or more terminal carbonyl groups are envisaged, wherein the carbonyl carbon is covalently attached to (a) the core structure, and (b) an X group of the first generation of the dendrimer, as follows:

core-C(═O)—X

Similarly, oxygen core functional atoms may be part of carboxylic acid, ether or ester groups of the core structure, or part of other groups derived from the oxygen-containing functional groups defined herein, which groups are bonded to or part of the core structure, wherein the oxygen core functional atom is covalently attached to an X group of the first generation of the dendrimer.

Sulphur core functional atoms may be part of sulphur dioxide, —S(═O)₂—, groups for example, or other groups derived from one of the sulphur-containing functional groups defined herein. The group is bonded to or part of the core structure, and core structures similar to those listed above, except having terminal sulfur-containing groups, are envisaged, the sulphur atoms being bonded to an X group of the first generation of the dendrimer.

Preferably, X is either selected from unsubstituted, uninterrupted C₁₋₁₆ alkylene groups (an example being a polyalkylenimine dendrimer such as a PPI dendrimer, or a DAB PPI dendrimer); or selected from C₁₋₁₆ alkylene groups interrupted with an N(R²) group and containing an oxo substituent (an example being a PAMAM dendrimer).

X may be selected from groups having the general structure Cₚ alkylene-C(O)N(R²)—C_{q} alkylene wherein p and q are integers and p+q is in the range 2 to 16. In this case, X is preferably selected from groups having the general structure C₁₋₆ alkylene-C(O)NH—C₁₋₆ alkylene.

Alternatively, X may be selected from linear unsubstituted C₁₋₁₆ alkylene groups. In this case, X is preferably selected from ethylene, propylene, butylene, pentylene and hexylene.

Preferably, X is the same group in each and every generation of the dendrimer. However, alternative embodiments are envisaged wherein X differs between different generations of the dendrimer, so that X in a particular generation is different from X in a subsequent generation. However, X is generally the same throughout any one particular generation.

Most preferably, X is either —(CH₂)₂—C(═O)N(H)—(CH₂)₂— (e.g. in a PAMAM dendrimer) or propylene (in a PPI dendrimer).

Preferably T is H or C₁₋₄ alkyl, so that the terminal groups of the dendrimer are NH₂ or N(R⁴)₂ wherein R⁴ is C₁₋₄ alkyl. Even more preferably, T is H or methyl, so that the terminal groups of the dendrimer are NH₂ or NMe₂.

The nitrogen-containing groups of the compound of formula III may be in a cationic, quaternary form. Preferably substantially only terminal amino groups of the dendrimer are in a quaternary form. Preferably, the terminal amino groups in the quarternary form comprise three C₁₋₄ alkyl groups covalently bound to the nitrogen atom of the terminal amino group. More preferably said C₁₋₄ alkyl groups are methyl groups, so that the terminal groups are —N⁺Me₃.

The compound of formula III may be a polyamidoamine (PAMAM) dendrimer wherein n is in the range 1 to 6.

T may be selected from amidoethylethanolamine, hexylamide, succinamic acid, Tris(hydroxymethyl)amidomethane, amidoethanol, amino and carboxylate groups.

A preferred compound of formula III is SuperFect, which is available commercially from Qiagen.

Alternatively, the compound of formula III may be a poly(propylenimine) dendrimer having a 1,4-diaminobutane core.

Compounds for use in the second aspect of the invention include activated or fractured (e.g. heat fractured) derivatives of the dendrimer compounds of formula III or formula IV. These derivatives include activated SuperFect or fractured SuperFect, which is commercially available from Quiagen.

Preferably, T is either H or methyl.

Preferably, when the compound of formula III is a poly(propylenimine) dendrimer wherein n is 2 (e.g. DAB8) T is methyl and the terminal amino groups are in the cationic quaternary form comprising three methyl groups covalently bound to the nitrogen atoms of said amino groups. It is particularly preferred that DAB8 is used in the present invention in the quaternary form, thus QDAB8 is more preferable than DAB8. This is because quaternised DAB8 has a lower general in vivo toxicity than non-quaternised DAB8.

Preferably the compound of formula III or salt thereof is not complexed to a nucleic acid molecule.

Preferably, the compound of formula III or salt thereof is not complexed to a therapeutic agent.

Preferably, the compound of formula III or salt thereof is not complexed to an agent that is active for the treatment of a condition characterized by undesirable cellular proliferation.

Preferably, the compound of formula III or salt thereof is not conjugated, completed, coupled, bonded, or non-covalently associated with one or more glucosamine or glucosamine-6-sulphate molecules. Preferably, the compound of formula III or salt thereof is not conjugated, completed, coupled, bonded or non-covalently associated with one or more naphthyl 3,6-disulfonic acid groups.

Preferably, in formula I of the first and third aspects of the invention, said C₁₋₁₆ alkyl and C₁₋₁₆ alkylene groups are optionally substituted by one or more groups selected from oxo, amino, hydroxy, carboxy, alkoxy, ester and halo.

Preferably, A and A′ are selected from unsubstituted C₁₋₆ alkylene groups. More preferably, A and A′ are ethylene.

Preferably, the B groups of the backbone monomer units are independently selected from H and a branching group of formula II. Similarly, the B′ groups of the monomer units of the branching group are preferably independently selected from H and a branching group of formula II.

R′ and R″ may be selected from unsubstituted C₁₋₆ alkyl groups. Preferably, R′ and R″ are selected from H, methyl and ethyl.

Preferably, R is selected from H and NR²R³ wherein R² and R³ are H or unsubstituted C₁₋₆ alkyl groups. More preferably, R is selected from H, NH₂, NMe₂ and NEt₂.

Preferably, the compound of formula I has an overall cationic charge (i.e. it is positively charged overall) at physiological pH.

This overall cationic charge arises as a result of the polymer containing nitrogen atoms at various positions therein, including within terminal amino groups, e.g. L-NH₂ or L-NR′₂ and/or within internal groups (denoted “internal nitrogen-containing groups”) such as groups interrupting an alkyl or alkylene group within a linear part of the polymer structure, e.g. L-N(H)-L′ or L-N(R′)-L′; or at the intersection of a polymer branch, e.g. L-N(-L′)-L″, wherein L, L′ and L″ may be alkylene groups as defined herein, and R′ may be an alkyl group as defined herein, for example.

The terminal amino groups and/or internal nitrogen-containing groups preferably have pKa's which cause them to be protonated, and therefore cationic, at physiological pH. Preferably, the terminal amino groups and/or internal nitrogen-containing groups of the compound of formula I have pKa's above 7, more preferably above 7.5, and most preferably in the range 8 to 12.

However, it may be that only terminal amino groups of the polymer (and not internal nitrogen-containing groups) have such preferable pKa values. Indeed, the pKa values of terminal amino groups would generally be expected to be within the preferred pKa range, and hence protonated and cationic at physiological pH. This is exemplified by the pKa values listed above (all in the range 9-11) of α-NH₃⁺ groups of amino acids.

Thus, it is preferred that the terminal groups of the compound of formula I (i.e. groups that are situated at the ends of the polymer including at the ends of polymer branches, and substituents of such groups) are predominantly cationic at physiological pH. Preferably these groups have pKa's above 7, more preferably above 7.5, and most preferably in the range 8 to 12. Preferably, these terminal groups include amino groups.

The nitrogen-containing groups of the compound of formula I (including internal nitrogen-containing groups and terminal amino groups) may be in a cationic, quaternary form. However, it may be that substantially only the terminal amino groups of the compound of formula I are in a quaternary form.

The terminal amino groups in the quarternary form may comprise three C₁₋₆ alkyl groups covalently bound to the nitrogen atom of the terminal amino group. Preferably, said C₁₋₆ alkyl groups are methyl groups.

The compound of formula I may be a polyethylenimine compound.

The compound of formula I may have a molecular weight in the range 0.6 kD to 800 kD, e.g. in the range 5 to 45 kD, or in the range 21 to 24 kD. In certain embodiments, for example when the compounds is linear polyethyleneimine, it may have a molecular weight of 22 kD.

In the first aspect of the invention it is preferred that n, which denotes the number of backbone monomer units -[A-N(B)]— in the compound of formula I, is greater than or equal to 20. It is more preferred that n is greater than or equal to 25. It is even more preferred that n is greater than or equal to 30, 50, 75, 100, 150 or 200, in order of increasing preference.

In the first aspect of the invention, it is preferred that n, which denotes the number of backbone monomer units -[A-N(B)]— in in the compound of formula I, is less than or equal to 20000. It is more preferred that n is less than or equal to 10000. It is even more preferred that n is less than or equal to 5000, 1000, 800 or 700, in order of increasing preference.

Thus, in the first aspect of the invention there are preferred ranges for n, determined by any combination of the preferred maximum and minimum values for n outlined above.

Preferably, in the first aspect of the invention, the compound of formula I or salt thereof is not complexed to a nucleic acid molecule.

Preferably, in the first aspect of the invention, the compound of formula I or salt thereof is not complexed to a therapeutic agent.

Preferably, in the first aspect of the invention, the compound of formula I or salt thereof is not complexed to an agent that is active for the treatment of a condition characterized by undesirable cellular proliferation.

When used in the compositions of the third aspect of the invention, n, which denotes the number of backbone monomer units -[A-N(B)]— in the compound of formula I, is preferably less than or equal to 20000. It is more preferred that n is less than or equal to 10000. It is even more preferred that n is less than or equal to 5000, 1000, 700, 500, 300, 250, 200, 150, 125, 100, 75, 50 or 30 in order of increasing preference.

Thus, preferred ranges for n in the compound of formula I when used in the compositions of the third aspect of the invention are 3-20000; 3-10000; 3-5000; 3-1000; 3-700; 3-500; 3-300; 3-250; 3-200; 3-150; 3-125; 3-100; 3-75; 3-50 or 3-30 in order of increasing preference.

In the compounds of formula I it is preferred that the average value for m, which denotes the number of monomer units -[A′-N(B′)]— in a branching group of formula II, is less than 0.5 n, where n denotes the number of backbone monomer units -[A-N(B)]— in the compound of formula I. It is more preferred that the average value for m is less than 0.25 n. It is even more preferred that the average value for m is less that 0.1 n. It is most preferred that the average value for m is less than 0.01 n. This is because it is preferable that the compound of formula I is substantially linear. The “average value for m” means the mean number of repeat units m in a branching group, taking into account all the branching groups (of formula II) within the compound of formula I. It is preferred that m is only a small fraction of n, because the compound of formula I is preferably substantially linear.

Preferably, the compound of formula I is substantially linear, wherein the branching groups of formula II are located on average, at every qth nitrogen atom along any given polymer chain segment, wherein q is greater than 3 or greater than 3.5. More preferably, q is greater than 10.

In this case, substantially all (e.g. above 80%, preferably above 90%, more preferably above 95%, and most preferably above 98%) of the B groups of the backbone monomer units may be H, and substantially all (e.g. above 80%, preferably above 90%, more preferably above 95%, and most preferably above 98%) of the B′ groups of the branching group of formula II may be H.

Preferably, the compound of formula I is not a dendrimer.

### Conjugates

The polymers and dendrimers for use in the present invention, including those of formulae I, III and IV described herein, may be associated with one or more molecules or ligands. This may be in order to improve the biodistribution, bioavailability, biocompatibility and/or physiochemistry of the polymer, for example. The term “associated with”, as used herein, includes covalent conjugation, either directly or via a linker or tether molecule, as well as non-covalent association or complexation (e.g. by electrostatic or other non-covalent interaction).

In particular, the polymers described herein may be associated with molecules or ligands that facilitate in vivo targeting of the polymer (“targeting moieties”). Thus, the polymers of the invention may be targeted to tumours by association (e.g. by covalent linkage, or electrostatic association) with a ligand capable of binding to a receptor (e.g. a protein) on the surface of a given tumour.

Various strategies for targeting tumours in this way are known to those skilled in the art, as described by Cassidy, J. and A. G. Schatzlein (2004) “Tumour targeted drug and gene delivery: principles and concepts.” Expert Reviews in Molecular Medicine in press, and by Schätzlein, A. G. (2003) “Targeting of synthetic gene delivery systems.” Journal of Biomedicine and Biotechnology 2003(2): 149-158.

### Hyaluronic Acid Conjugates

A preferred moiety for facilitating in vivo targeting of the polymeric compounds of the invention is hyaluronic acid (HA). The polymers of formulae I, III and IV described herein may be associated with hyaluronic acid (HA). HA is an anionic polysaccharide composed of repeating units of beta-1-4-glucuronate-beta-1-3-N-acetylglucosamine, as shown below:

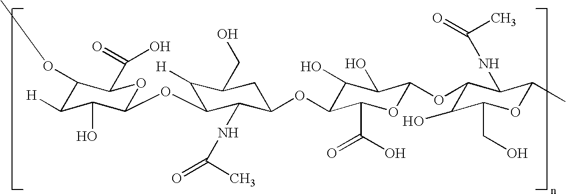

Hyaluronic acid is the natural ligand of the CD44 receptor which is overexpressed in a number of tumours but has also been implicated as a marker for cancer stem cells [56]. Thus, HA is capable of selective binding to such tumours in which CD44 is overexpressed, and may be used to target the polymers in the present invention to the tumours.

Preferably, the polymer compound of formulae I, III or IV is linked to HA through covalent conjugation of the polymer to the HA backbone. Preferably the polymer compound of formulae I, III or IV is linked to low molecular weight HA. Low molecular weight HA may be produced by acid hydrolysis or enzymatic cleavage (see below). Preferably, the covalent linkage between HA and the polymer is via an amide bond C(═O)—N(H). Preferably, the amide bond is formed through reaction of a terminal amino group of the polymer with a carboxyl group of HA. Preferably, 1-ethyl-3-(3-dimethylaminopropyl) carbodiimide (EDAC) is used as a coupling reagent to activate the carboxyl group of HA for coupling with a terminal primary amino group of the polymer, forming an amido linkage between HA and polymer.

While an amido linkage between HA and the polymer is exemplified, other types of covalent linkages between HA and the polymers of the invention are envisaged. Various covalent linkages between polymer and HA may be created using standard coupling chemistry, as would be appreciated by the skilled person. For example, a carboxyl group of HA may be reacted with a different, suitable substituent group on the polymer (e.g. a substituent group selected from those defined hereinbefore, such as a hydroxyl group) to covalently link the two molecules. Alternatively, the carboxyl groups of HA may first be derivatised to form other reactive functional groups (e.g. acid amide or acid chloride groups) that may then be reacted with a suitable substituent (e.g. selected from those defined above) on the polymer.

Although direct covalent coupling of HA to the polymers is an option, a tether or linker molecule may be used. The tether or linker may itself be a biocompatible polymer or oligomer such as poly(ethylene glycol) (PEG), or a polyethylenimine polymer or oligomer, or another linker molecule such as an optionally substituted, optionally interrupted alkylene chain. The skilled person would be aware of suitable linker molecules. Again, standard coupling chemistry could be used to couple each end of the linker molecule to HA and a polymer of the invention respectively. Preferably the linker molecule is PEG.

The polymers of the present invention may be derivatised by covalent attachment of PEG chains thereto, as exemplified in Brownlie, A., I. F. Uchegbu and A. G. Schatzlein (2004) “PEI-based vesicle-polymer hybrid gene delivery system with improved biocompatibility.” Int J Pharm 274(1-2): 41-52, which describes the covalent coupling of PEG chains to branched polyethylenimine to form comb-type co-polymers. See also Luo et al., Macromolecules 2002, 35, 3456-3462, which describes the synthesis of PEG-conjugated PAMAM dendrimer. Thus, one or more of these PEG chains may be used as a linker molecule for coupling the polymer to a targeting ligand such as HA. Indeed, the “free end” of a PEG chain in such a comb-type copolymer could be coupled (using standard coupling chemistry) to HA. Of course, reaction of the PEG terminus of a comb-type polymer with an HA molecule would be facilitated by the use of (hetero-) bifunctional PEG in forming the comb-type polymer, so that the PEG terminus was suitably functionalised (e.g. with a terminal amino group) for reaction with HA. Alternatively, the comb-type polymer itself could be further derivatised so that the PEG terminus comprised a functional group (such as an amino group) suitable for reaction with HA (e.g. in the presence of the coupling agent EDAC). Linkers have been used previously to target polyamino-polymers (see Brown, M. D., A. I. Gray, L. Tetley, A. Santovena, J. Rene, A. G. Schatzlein and I. F. Uchegbu (2003). “In vitro and in vivo gene transfer with poly(amino acid) vesicles.” J Control Release 93(2): 193-211).

While covalent linkage of the polymers of formulae I, III and IV to HA is preferred, complexation through non-covalent (e.g. electrostatic) interactions is also envisaged.

### Other Ligands

Association of the polymers described herein with ligands other than HA is also envisaged. For example, protein or carbohydrate ligand or another type of polymeric ligand may be associated with these polymers. As described above for HA, the linkage may be covalent, e.g. via a linker or tether molecule, or non-covalent, e.g. electrostatic. Thus, a protein ligand for, or antibody against, any receptor or other molecule expressed on the surface of a tumour cell (e.g. a tumour-specific antigen), may be associated with a polymer described herein, to facilitate targeting of that polymer to the tumour cells. A number of different types of ligands could be coupled to the polymer in this way (possibly in combination with each other, or in combination with HA—see below).

The targeting moieties may be endogenous or exogenous, synthetic or naturally occurring. Naturally-occurring ligands which may be coupled to the polymers described herein include small molecules, such as biotin-avidin, and folate receptor/folate. Other peptides or proteins may be coupled to the polymers described herein, including phage-derived peptides, antibodies, antibody fragments, and endogenous peptides or proteins such as growth factors, hormones or any other molecule capable of binding specifically to a molecule expressed on the surface of the desired target cell type. Examples include EGF, transferrin, carbohydrates, lectins, polymeric molecules such as hyaluronic acid (HA), and antibodies and fragments thereof. Antibody fragments ideally retain antigen binding capability (e.g. Fab fragments) but may consist of or comprise constant regions of the molecule such as Fc domains, e.g. if the target cell carries Fc receptors.

Coupling strategies and chemistries suitable for associating the above ligands with the polymers described herein (either covalently or non-covalently) are apparent to the skilled person: some of these are described above in relation to HA.

### Combinations of Ligands

The polymers described herein may be associated with a plurality of different targeting moieties. Thus a polymer may be linked to a combination of the ligands or ligand types described above. This is useful for cross-sectional targeting of the polymers described herein. For example, if a first ligand binds a receptor on target tumour cells as well as a receptor on a first population of non-target cells, and if a second ligand binds a receptor on the same target cells as well as a receptor on another (second) population of non-target cells, then association of a polymer of the invention with both the first and second ligands can result in higher specificity of the polymer for the target tumour cells than for the each population of non-target cells.

### Reversible Coupling of Ligands

The association (whether by covalent coupling or electrostatic attraction) of the ligands described above (e.g. HA) with the polymers described herein may be reversible, or cleavable. For example, a cleavable covalent linker (or alternatively a “% reversible” electrostatic attraction) may be employed, which reacts to environmental changes (e.g. pH, or hypoxia) to trigger release of the ligand from the polymer.

This is especially important if the polymer of the invention is inactive when bound to a targeting moiety, such that rescue of the activity of the polymer is required once the polymer has been successfully delivered to the target location.

Preferably, in this case, a cleavable covalent linker is used to link the targeting ligand to the polymer. Preferably, the polymer and targeting ligand become separated upon delivery of the polymer to the target. Preferably, the cleavable covalent linker reacts to an environmental change that occurs upon delivery of the polymer to the target location, causing separation of the polymer from the ligand. This environmental change may be a change of pH or hypoxia at the target location. Alternatively, cellular (e.g. endosomal) enzymes and/or extracellular enzymes (e.g. metalloproteinases) may trigger release of the polymer from the ligand. Thus, enzymes generated within target tumour cells could effect release of the polymer from the ligand, e.g. by cleavage of the ligand, allowing the polymer to become active and attack the tumour. A protease enzyme, for example, might cleave a peptide (amido) bond linking the polymer to the ligand. Such strategies are described in Damen, E. W., T. J. Nevalainen, T. J. van den Bergh, F. M. de Groot and H. W. Scheeren (2002). “Synthesis of novel paclitaxel prodrugs designed for bioreductive activation in hypoxic tumour tissue.” Bioorg Med Chem 10(1): 71-7.; Cassidy, J., R. Duncan, G. J. Morrison, J. Strohalm, D. Plocova, J. Kopecek and S. B. Kaye (1989). “Activity of N-(2-hydroxypropyl)methacrylamide copolymers containing daunomycin against a rat tumour model.” Biochem Pharmacol 38(6): 875-9; and de Groot, F. M., E. W. Damen and H. W. Scheeren (2001). “Anticancer prodrugs for application in monotherapy: targeting hypoxia, tumor-associated enzymes, and receptors.” Curr Med Chem 8(9): 1093-122.

Alternatively, the cleavable covalent linker may be photocleavable. This is especially useful if the polymer of the invention is inactive when conjugated to the targeting ligand, and active when released from the ligand. Thus, upon delivery of the polymer to the desired location (e.g. a particular tumour), the tumour can be irradiated in order to cleave the ligand from the polymer and render the polymer active at the site of the tumour.

The use of self-eliminating spacers, linking the polymer to the targeting ligand, may also be useful to reconstitue full activity of the polymer, as described in de Groot, F. M., C. Albrecht, R. Koekkoek, P. H. Beusker and H. W. Scheeren (2003). ““Cascade-release dendrimers” liberate all end groups upon a single triggering event in the dendritic core.” Angew Chem Int Ed Engl 42(37): 4490-4.

### Carriers and Nanoparticle Complexes

The targeting moieties described above may be associated (normally covalently but in principle also non-covalently) with a carrier, the carrier also being associated with a polymer used in the methods of the invention, so that the targeting moieties are presented near the surface of the carrier. This may facilitate interaction between the ligand and a ‘receptor’ that is complementary to the targeting ligand. Sometimes spacers or tethers are used (see above) to link the ligand to the particulate carrier in order to create a steric situation that allows easy access. The carrier may be a biocompatible polymer or other biomolecule, for example.

Thus the polymers (including dendrimers) used in the present invention, including those of formulae I, III and IV described herein, may be associated (e.g. covalently or electrostatically) with a carrier. Complexes between such polymers and carriers tend to form nanoparticles, which may be a convenient form for administration.

The carrier may be a biomolecule, e.g. a nucleic acid (typically DNA), or HA, as described above. The biodistribution, bioavailability, biocompatibility and/or physiochemistry of the polymer may be improved in such nanoparticle form.

A nucleic acid carrier as used in this aspect of the invention may be incapable of being expressed (i.e. transcribed and/or translated); thus when introduced into a target cell, it does not give rise to an RNA or protein expression product. For example, even if the nucleic acid contains an open reading frame, it may contain no promoter (e.g. a promoterless plasmid).

Alternatively, a polymer may be complexed into nanoparticle form by complexation with an active biomolecule, in which case the polymer and biomolecule complexed thereto may show synergistic effects. For example, a polymer may be complexed with a nucleic acid which is capable of being expressed (transcribed and/or translated), giving rise to a therapeutically active expression product such as a protein or RNA. For example, the carrier may be an expression vector encoding a therapeutically useful protein such as TNF.

The effects of complexing DAB16 to a promoterless plasmid and an expression plasmid carrying a strong promoter are described below and shown in FIG. 7.

### Bioactive Molecules

The bioactive molecule of the composition of the third aspect of the invention is preferably anionic at physiological pH, preferably carrying more than one negative charge per molecule, in order that the cationic groups of the polymer of formula I are able to form non-covalent electrostatic interactions with the bioactive molecule.

The bioactive molecule may itself be a polymer, such as heparin (a polyanion at physiological pH) or a related polymer, e.g. another polymer with a high level of anionic sulphate and/or carboxyl substituents. Alternatively, the bioactive molecule may be an extracellular matrix polymer such as dextran.

The bioactive molecule may be a peptide or protein. Peptides or proteins having pKa's such that they are negatively charged around physiological pH (such as anionic drug molecules) are particularly preferable.

For example, the bioactive molecule may be a polyanion which is a potent inhibitor of HIV, e.g. a negatively charged albumin, or dextran sulphate. Anionic albumins with potent anti-HIV activity are described at (http://www.niwi.knaw.nl/en/oi/nod/onderzoek/OND1270824/toon).

The bioactive molecule may be a conventional organic drug molecule, e.g. with one or more carboxylic acid groups that are negatively charged at physiological pH. Examples are diclofenace, phenobarbital and barbituric acid.

### Gene Delivery

Without wishing to be limited by any particular theory, it is believed that the polymers described herein may exert cytostatic effects on tumour cells in vivo. Thus cells treated with these polymers may not divide. Non-dividing cells are less sensitive to certain cytotoxic drugs than dividing cells of a similar type. Thus particular benefits may be achieved by using polymers as described above in relation to any aspect of the invention for specific types of gene therapy for diseases characterised by undesirable cellular proliferation, especially neoplastic disease such as cancers as described above.

Thus the polymers may be used for delivery of a nucleic acid (e.g an expression vector) encoding an enzyme capable of converting a prodrug to a more active, cytotoxic form, wherein the cytotoxic form is more toxic against dividing cells than against non-dividing cells.

Cells which receive the enzyme therefore become capable of converting prodrug to drug, but are prevented from proliferating by the cytostatic effects of the polymer delivery agent. Thus these cells become a source of active drug molecule while at the same time becoming more resistant to the effects of the drug than surrounding untreated cells. The life of the enzyme-carrying cells as a source of active drug molecule is therefore prolonged, potentially increasing the efficiency of the treatment. If and when the cytostatic effect wears off, the cells will be killed by the drug molecule, and thus should not be able to escape to allow tumour regrowth.

Examples of suitable drugs which are more active against dividing than non-dividing cells include nucleoside analogues such as 5-fluorouracil. Prodrugs include ganciclovir. Enzymes which may be used in conjunction with such prodrugs include thymidine kinase from Herpes Simplex Virus.

Thus the invention includes the use of a polymer as described above for the preparation of a composition for the delivery of a nucleic acid to a cancer cell, the nucleic acid encoding an enzyme capable of converting a prodrug to a more active, cytotoxic form, wherein the cytotoxic form is more toxic against a dividing cell than against a non-dividing cell.

### Hydrophobicity

The polymers used in the present invention can be modified by covalently binding derivatising pendant groups, such as hydrophobic or hydrophilic groups, to the surface of the dendrimer. A combination of hydrophobic and hydrophilic substituents may be attached to make hydrophilic polymers amphiphilic. Amphiphilicity allows for broad manipulation of phsyciochemistry, e.g. for self assembly (formation of polymeric vesicles, micelles, etc. and even hydrogels), which is useful for modification or optimisation of the in vivo properties of the polymer. The number of derivatising groups may vary from one derivatising group per polymer molecule up to and including derivatising all available surface or terminal groups, for example, derivatising all 8 surface groups of a DAB8 molecule or all 16 surface groups of a DAB16 molecule. Derivatising dendrimer molecules is described in WO 03/033027.

### BRIEF DESCRIPTION OF THE DRAWINGS

FIG. 1 shows cytostatic effects induced by various polymers in vitro.

FIG. 2 shows inhibition of tumour growth by four DAB dendrimer polymers, quaternarised DAB8, fractured SuperFect (PAMAM polymer) and linear PEI. Established experimental A431 murine xenografts (control=red) were treated by a single injection of the relevant polymer.

FIG. 3 shows body weight change in A4311-bearing mice. Untreated animals and animals treated with a single dose of the various polymers were weighed and changes expressed in percent change compared to the day of the first treatment.

FIG. 4 shows treatment of established LS174T Human Colorectal Adenocarcinoma (ATCC CCL-188) xenografts in a mouse model. One group of animals (black) was untreated. The remainder were treated (q.2d 5×) with either DAB16 polymer (green), naked plasmid encoding TNF alpha (red) and a complex of DAB16 and the TNF alpha-encoding plasmid (blue). Individual animals are represented by separate symbols.

FIG. 5 shows treatment of established C33a Human Cervix Carcinoma (ATCC HTB31) xenografts in a mouse model. Animals treated (q.2d 5×) with DAB16 (green) were compared to untreated animals (black), and those treated with naked plasmid encoding TNF alpha (red) or a DAB16-TNF alpha plasmid complex (blue). Individual animals are represented by separate symbols.

FIG. 6 shows treatment of established A431 epidermoid carcinoma (ATCC CRL-1555) in a mouse model. Animals treated (q.2d 5×) with DAB16 (green) were compared to untreated animals (black), and those treated with naked plasmid encoding TNF alpha (red) or a DAB16-TNF alpha plasmid complex (blue).

FIG. 7. A431 epidermoid carcinoma tumours were grafted into nude CD-1 mice and left to establish (˜5 mm). Animals were treated by injection of the relevant formulation every 2^{nd} day over 10 days (5 injections). The ability of the generation 3 polypropylenimine dendrimer (DAB16) as a single agent to delay long-term tumour growth (green) was compared with that of a naked TNF alpha-encoding plasmid (blue), a complex of both (magenta), DAB16 complexed to promoterless plasmid (cyan). Untreated control is shown in red. Tumour volume doubling time was measured as a surrogate endpoint as substantial tumour growth immediately precedes tumour related mortality. Complexes of DAB16 and non-functional DNA (a promoterless TNF alpha plasmid) as well as free dendrimer show improved long-term growth reduction.

FIG. 8 shows overall tumour response to treatment, stratified according to change in tumour volume into progressive disease (increase greater than 1.2 fold), stable disease (0.7-1.2), partial response (0-0.7), and complete response (0) over the duration of the experiment.

FIG. 9 shows activity and toxicity of doxorubicin in A431 xenograft models (taken from [55]).

FIG. 10 shows that hyaluronic acid conjugates of DAB16 (HA-dendrimer) can target cancer cells expressing the CD44 receptor. Complexes formed from plasmid DNA and conjugates of HA-dendrimer show superior targeting to CD44 positive cells as compared to complexes formed with un-conjugated dendrimer [57, 58].

FIG. 11 shows that HA-dendrimers preferentially target plasmid encoding beta-galactosidase to CD44 positive B16F10 melanomas in vivo, in contrast to unconjugated linear PEI (“Polymer”) [57, 58].

### EXAMPLES

The following compounds were obtained from commercial sources: DAB4, DAB8, DAB16, DAB32, DAB64, SuperFect, linear polyethylenimine (22 kD).

Hyaluronic acid (HA) conjugates of DAB8 (generation 2 PPI dendrimer) and DAB16 (generation 3 PPI dendrimer) were synthesized according to the procedure outlined below.

Quaternised DAB8, DAB16, DAB32 and DAB64 (termed QDAB8, QDAB16, QDAB32 and QDAB64) were synthesized according to the method below, in which each of the nitrogen atoms of the terminal amino groups of these dendrimers is converted to a cationic quaternary ammonium group having three methyl groups bonded to the nitrogen atom.

### Synthesis of Targeted Hyaluronic Acid DAB Dendrimers

Low molecular weight hyaluronic acid was synthesized by heat or enzyme degradation, as follows:

### Heat Degradation (HA24, HA48)

500 mg hyaluronic acid (500 mg) was added to acid buffer solution [tri-hydroxy methyl-amino methane (0.1M), potassium chloride (0.1M), monobasic potassium phosphate (0.1M), anhydrous citric acid (0.1M), sodium tetraborate (0.1M), pH=3, 100 ml] and subsequently degraded either 24 h or 48 h at 70 C.°. Degraded polymer samples were isolated by exhaustive dialysis against distilled water (5 L) with 6 changes over a 24 h period by using dialysis tubing with a molecular cut off of 12-14 KD. The dry solid was obtained by freeze-drying the dialysate.

Enzymatic degradation (HAenz)

Hyaluronic acid (HA, 1 g) (Scheme 1) was dissolved in phosphate buffer saline (PBS, ph=7.4, 300 ml) by stirring overnight at room temperature. A solution of bovine testis hyaluronidase was prepared by dissolving this enzyme (100 mg) in PBS (10 ml). Hyaluronic acid solution was heated for 30 min at 37 C.° in water bath and then the enzyme solution was added to the warm solution and the enzyme hyaluronic acid solution was heated for 48 h at 37 C.°. At the end of this time period the solution was boiled for 15 minutes to denature the hyaluronidase. The solution was allowed cool and then centrifuged (6000 rpm, 30 min). The precipitated enzyme was filtered out and then polymer solution was isolated by exhaustive dialysis against distilled water (5 L) with 6 changes over a 24 h period by using dialysis tubing with a molecular cut off of 12,000-14,000 Daltons. The dry solid was obtained by freeze-drying the dialysate.

The HA-DAB8 conjugates were then synthesized as follows:

### HA-DAB8 Conjugates

DAB8 was conjugated with HA24, HA48 and HAenz. Synthesis of these HA-DAB8 conjugates was carried out as depicted in Scheme 2, by reaction of DAB8 with low molecular weight hyaluronic acid (either HA24, HA48 or HAenz) in the presence of 1-ethyl-3-(3-dimethylaminopropyl) carbodiimide (EDAC) at a pH of 4.75.

EDAC is a well known carboxyl activating agent for amide bonding with primary amines, and may be used to link a biological substance containing a carboxylate group (such as HA) with a biological substance containing a primary amine (such as a DAB polypropylenimine dendrimer).

Either HA24, HA48 or HAenz (378 mg, 1.0 mmoles carboxylic acid groups) were dissolved in water (100 ml). Solid poly propylenimine octa amine dendrimer (DAB8, generation 2, 7.73 g, 10 mmoles, 7.73 ml) was added to the HA solutions. The pHs of the solutions were adjusted to pH 4.75 by addition of 0.1M HCl. Solid 1-ethyl-3-(3-dimethylaminopropyl) carbodiimide (EDAC) (1.92 g, 10.0 mmoles) was added to the acid reaction mixtures. The reactions were allowed to proceed for 2 h with stirring, the pHs adjusted upwards with NaOH (0.1M) to pH=7 and the products isolated by exhaustive dialysis against distilled water (5 L) with 6 changes over a 24 hour period by using dialysis tubing with a molecular cut off of 12-14 kD. The dry solids were obtained by freeze-drying the dialysates.

Hyaluronic acid (HA) conjugates of DAB16 were synthesized using similar procedures.

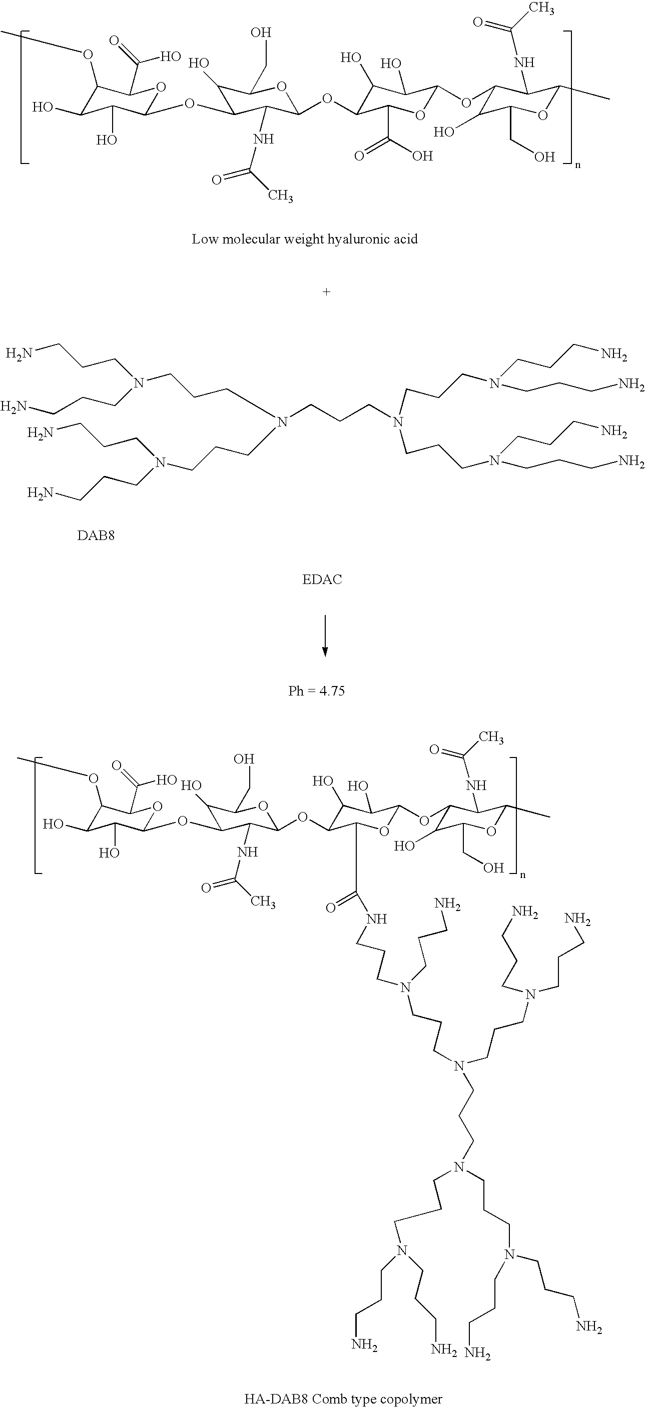

### Synthesis of Quaternised DABs

Synthesis was carried out as depicted in Scheme 3. For the quaternarisation of DAB polymers, DAB8 (generation 2), DAB16 (generation 3), DAB32 (generation 4) or DAB64 (generation 5) (500 mg, Sigma-Aldrich, UK) was dispersed in N-methyl-2-pyrrolidone (50 mL, Sigma-Aldrich, UK) for 16 h at room temperature by stirring. To the DAB dispersion was added sodium hydroxide (120 mg, Merck Eurolab, UK), methyl iodide (3 g, Sigma-Aldrich, UK) and sodium iodide (150 mg, Sigma-Aldrich, UK). The reaction mixture was stirred under a stream of nitrogen gas for 3 h at 36° C. The quaternary ammonium product (QDAB8, QDAB16, QDAB32 or QDAB64, obtained from DAB8, DAB16, DAB32 or DAB64 respectively) was then recovered by precipitation with diethyl ether (500 mL, Merck Eurolab, UK) followed by filtration.

The resulting solid was first quickly washed with absolute ethanol (1 L, Merck Eurolab, UK) over a vacuum pump, followed by diethyl ether (500 mL). The washed solid (quaternary ammonium product) was subsequently dissolved in water (150 mL) and passed over an Amberlite anion exchange column. The eluate obtained was freeze dried and obtained as a yellow solid, and the structure was confirmed by both ¹H and ¹³C NMR.

The Amberlite anion exchange column was prepared by placing Amberlite IRA-93 Cl (Merck Eurolab, UK) in a 100 mL separatory funnel and washing the resin first with HCL (1 M, 90 mL) followed by distilled water (500 mL) until the eluate gave a neutral pH.

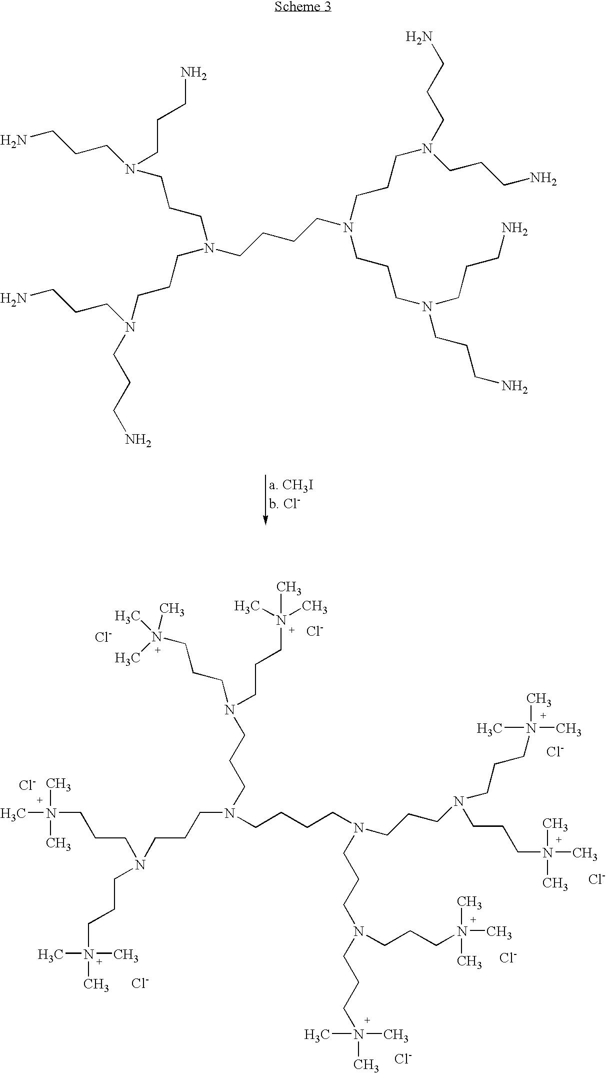

### In Vivo Experiments

### Animals

Female mice (CD1-nu, initial mean weight 20 g) were housed in groups of five in suspended plastic cages at 19-23° C. with a 12 h light-dark cycle. A conventional diet (Rat and Mouse Standard Expanded, B and K Universal, Grimston, UK) and water from the mains were available ad libidum. Experimental work was carried out in accordance with UK Home Office regulations and approved by the local ethics committee.

### Tumour Implantation

Tumour cells [LS174 Human Colorectal Adenocarcinoma (ATCC CL-188), A431 Epidermoid Carcinoma (ATCC CRL-1555), C33a Human Cervix Carcinoma (ATCC HTB31)] were grown as monolayers in 75 cm² flasks in Dulbecco's Modified Eagle's Medium (DMEM) supplemented with 10% (v/v) foetal bovine serum (FBS) and 1% (v/v) glutamine, in a humid atmosphere of 5% CO₂ at 37° C. Medium was changed twice a week. Cells were subcultured every seven days by trypsin treatment and experiments were conducted when the cells were in exponential phase. Nude mice were injected subcutaneously with the cell suspension in either flank and cells were then left to develop palpable tumours (typical diameters 5-6 mm); in every case 1×10⁶ cells were injected in each flank and tumours developed over 7 days (LS174T) to 10 days A431, C33a).

### Formulations

All formulations were prepared as solutions (or suspensions) in 5% dextrose. Each dose contained 250 μg of DAB 4, DAB 16, DAB 32, QDAB8, respectively. The PAMAM dendrimer and linear PEI were given as dilutions of Superfect (100 μl per animal) and Exgen (9 μl per animal) respectively, in 5% dextrose solution. Control formulations containing PP1-G3 (DAB16) polymers complexed with plasmid DNA (mTNFalpha expression vector (pORF9-mTNFα with a strong promoter (EFlalpha/HTLV or promoterless) and free TNFalpha plasmid were also prepared in 5% dextrose. Colloidal dispersions were sized by photon correlation spectroscopy (Malvern Zetasizer 3000, Malvern Instruments, UK).

### Experimental Therapy

Animals were injected intravenously (0.2 ml per injection) with the different formulations either once or alternatively on a schedule every other day (0.5 q.d.) over 10 days (every second day, 5 injection). Mice which did not received any treatment served as controls. Each group consisted of 5 animals (n=5). As a control DAB 16-DNA complexes were prepared as previously described [36] by mixing dendrimer and DNA (50 μg) at a 5:1 weight ratio in a 5% dextrose solution (200 μl/animal). Free plasmid DNA (50 μg) was given in 200 μL 5% dextrose. Animals were monitored at regular intervals, the tumour size was determined using callipers, and body weight measured and recorded. Expression of genes containined on nucleic acids complexed with the various polymers was measured as described previously [54].

### Results

Examples of active polymers include large fractured PANAM dendrimers (Superfect-L MW ˜35 kD), linear polymers (Exgen, 22 kD), and small dendrimers such as lower generation polypropylenimine dendrimers (DAB4-DAB64). These exhibit cytostatic effects towards tumour cell lines in vitro.

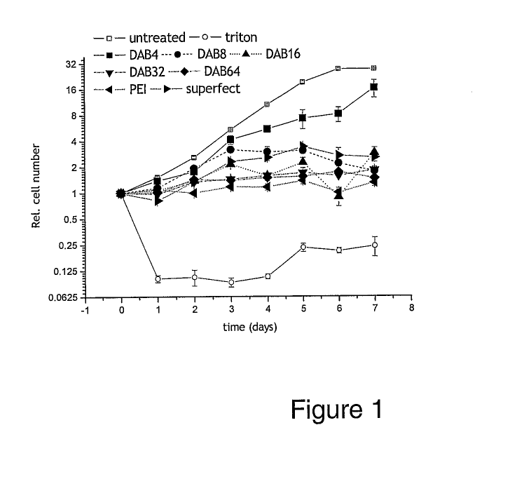

A431 epidermoid carcinoma cells were treated with various cationic polymers. PEI, Superfect and various DAB polymers were added to the culture medium at concentrations of 0.45 μL/mL, 5 μL/mL and 12.5 μg/mL respectively for the duration of the experiment. Untreated cells show typical growth behaviour; triton X treated cells show decrease in cell number consistent with cell lysis. The cytostatic effects on the tumour cell lines are illustrated in FIG. 1.

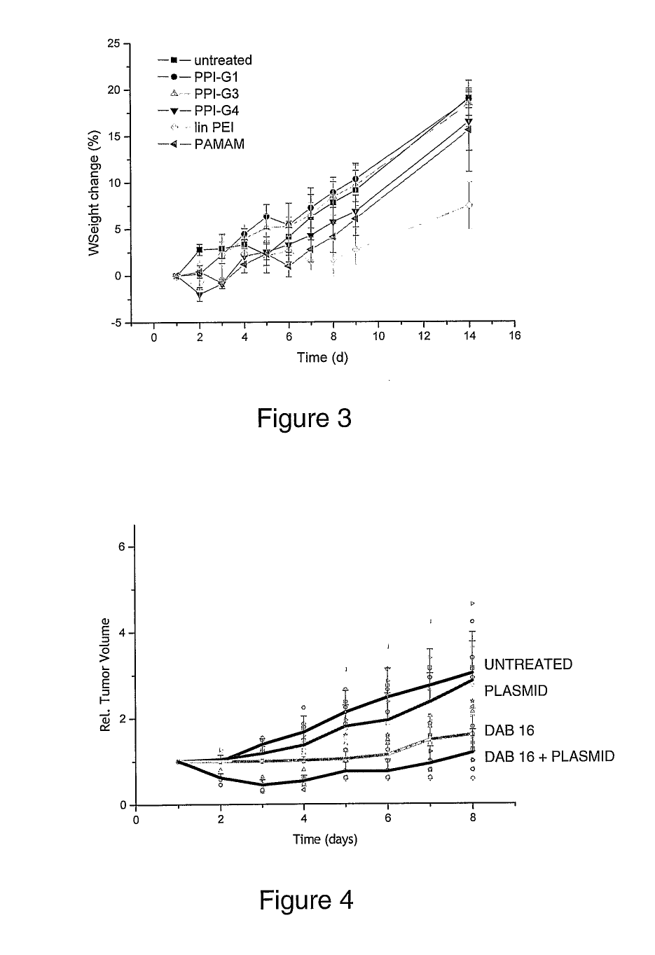

Polymers were then administered in vivo. Administration was at levels which we would expect to complex similar amounts of DNA, not at levels calculated to provide similar cytostatic effects. The effect is essentially the same for all materials so it is conceivable that the ability of these materials to bind DNA plays a role in the effects observed, e.g. through condensation of nuclear DNA. All polymers used were well tolerated with no apparent signs of gross, systemic toxicity in vivo (FIG. 3).

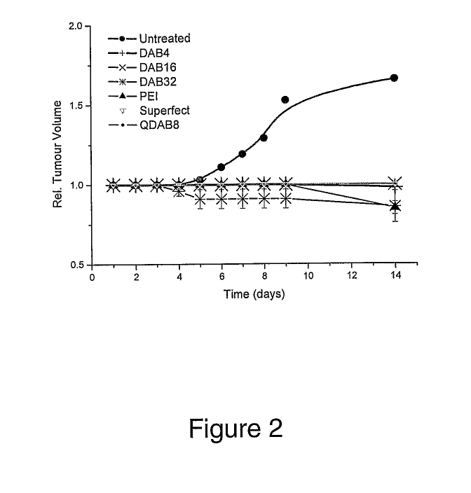

DAB8 (PPI G2) kills animals within 5-10 seconds after i.v. injection; however no such effect has been observed with any of the closely related DABs. By contrast the modified (quaternised) QDAB8 is well-tolerated and active (FIGS. 2 and 3). Therefore this effect is thought to be unique to underivatised DAB8.

When administered systemically to treat established A431 xenograft tumours all the polymers completely inhibit tumour growth and in some cases lead to a small reduction in tumour volume within the first two weeks (DAB32, PEI; cf. FIG. 2) while the untreated tumour grows unchecked.

Importantly there is no apparent systemic toxicity in vivo associated with this highly efficacious treatment. The animals are young and continue to grow during treatment. This is reflected in the increased body weight for all the formulations (FIG. 3) with the possible exception of PEI-treated animals for which the decrease is 5-10% less than for the other groups.

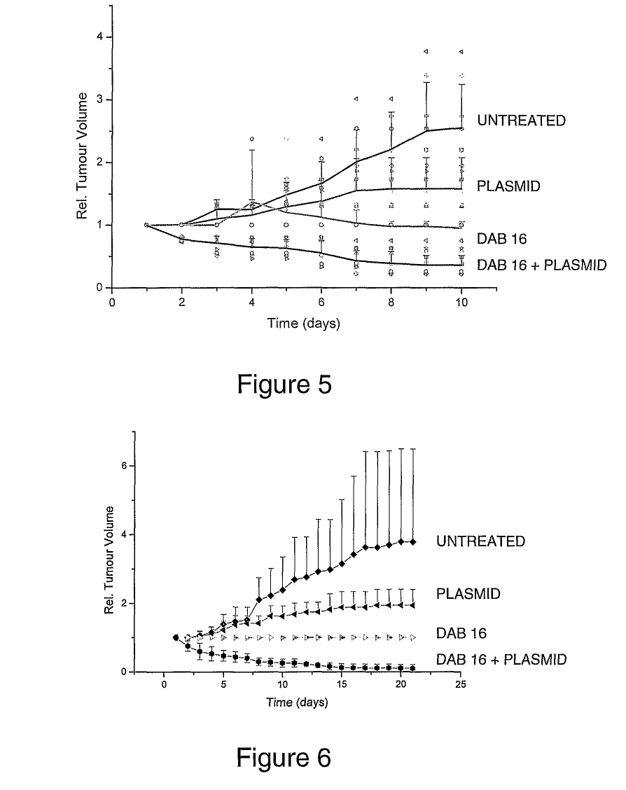

The effect is not unique for a specific tumour but was also observed in a number of xenograft models. Here the effect of the G3-PPI solution was compared with PP1-G3 DNA complexes carrying an expression plasmid for the murine TNFalpha gene (50 μg DNA complexed at 5:1 (w/w)) and the free TNFalpha plasmid (50 μg/animal) in established LS174T colorectal tumours (FIG. 4), C33a cervix carcinomas (FIG. 5), and the A431 epidermoid carcinoma model (FIG. 6). In each of the tumour models the treatment of animals with DAB16 inhibited tumour growth significantly.

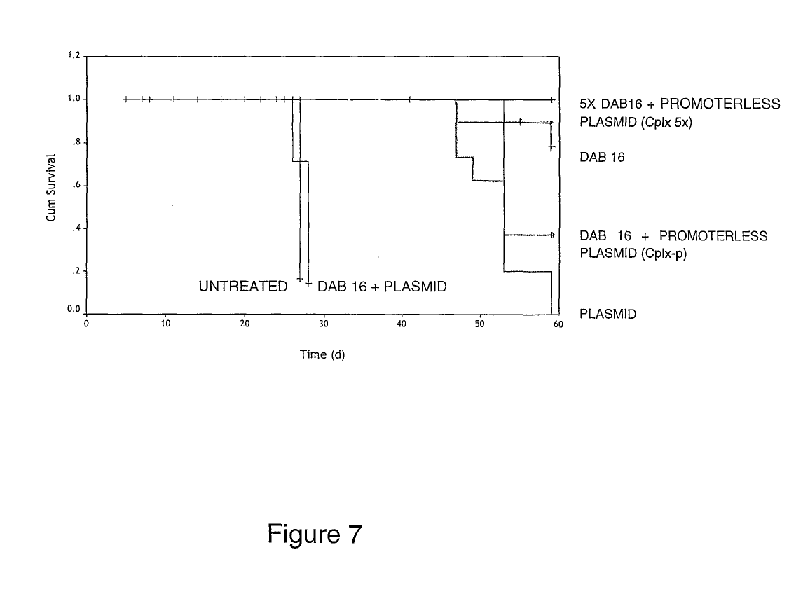

In a long term experiment the repeated administration of DAB16 (0.5 q.d. X₅) resulted in a decrease of the tumour size from day 23 for 2 mice, from day 33 for all the mice. The tumours even completely disappeared from day 43 and 51 for 2 mice (n=5) and resulted in long term survival of the treated mice (cf. FIG. 7).

The effect of the cationic polymers does not only depend on the injection of the free compound but is also seen when the compound is given in the form of nanoparticles (FIG. 7). Both free polymers and those complexed into nanoparticles through complexation with a promoterless plasmid (“Cplx −p”) were beneficial. Nanoparticles formed from the expression plasmid carrying a strong promoter and the dendrimer were highly active and showed synergistic effects (“Cplx 5×”). In contrast no beneficial effect was observed when the PPI-G3 and the plasmid were administered separately (“DAB+TNF”).

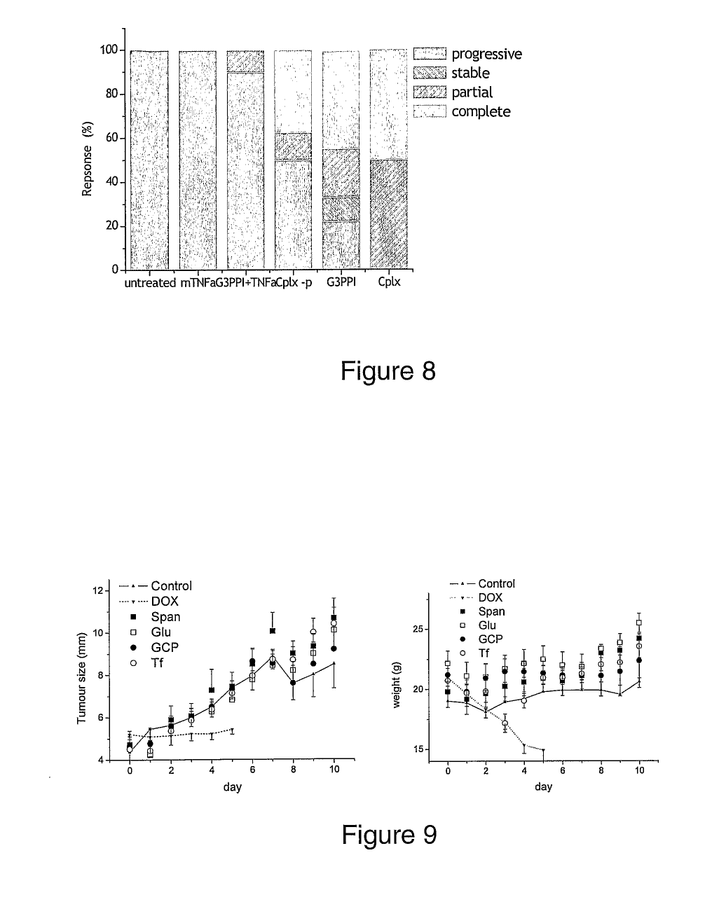

Overall tumour response to treatment was stratified according to change in tumour volume, into progressive disease (increase greater than 1.2 fold), stable disease (0.7-1.2), partial response (0-0.7), and complete response (0), over the duration of the experiment (12 weeks) analogous to the RECIST criteria (Therasse, P., S. G. Arbuck, et al. (2000). “New guidelines to evaluate the response to treatment in solid tumors. European Organization for Research and Treatment of Cancer, National Cancer Institute of the United States, National Cancer Institute of Canada.” J Natl Cancer Inst 92(3): 205-16.) The results of this analysis are shown in FIG. 8.

The magnitude of the effect of the cationic polymers alone is similar to that seen with the cytotoxic drug doxorubicin in the same tumour model (FIG. 9).

The polymers may also be targeted to tumours by association with a ligand capable of binding to a receptor (e.g. a protein) on the surface of a given tumour. Active targeting of DAB16 and DAB8 was achieved through conjugation of the appropriate dendrimer to a hyaluronic acid (HA) backbone. Low molecular weight HA was produced by acid hydrolysis or enzymatic cleavage and coupled to the dendrimers as described earlier. Hyaluronic acid is the natural ligand of the CD44 receptor which is overexpressed in a number of tumours but has also been implicated as a marker for cancer stem cells [56].

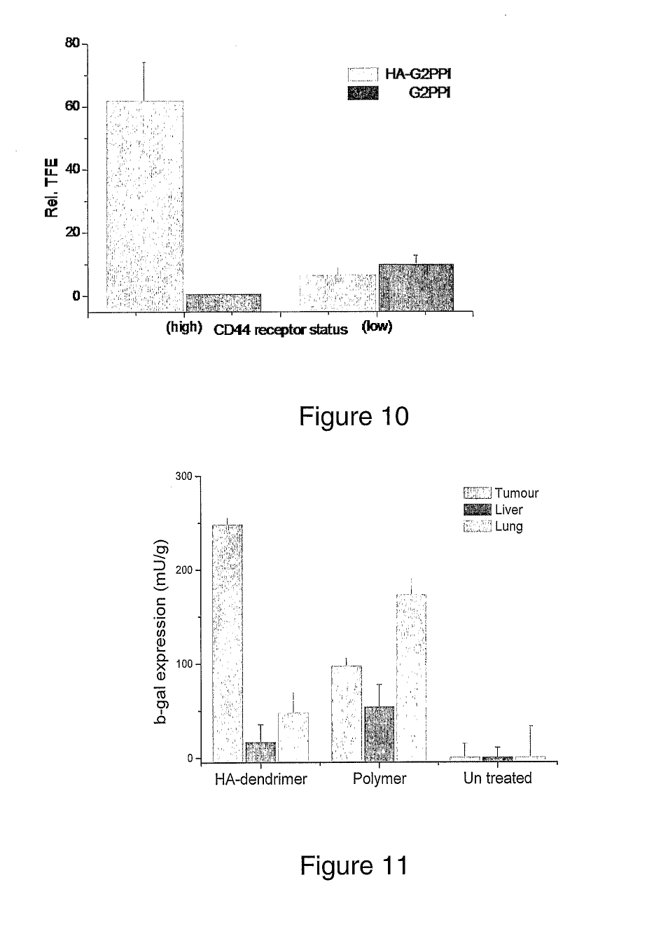

DNA complexes formed with the targeted polymers show preferential uptake in receptor positive cancer cells (B16F10 murine melanoma) but not in the control cells (NIH 3T3; FIG. 10). The targeted complexes also show a higher expression in the receptor positive tumours in the syngeneic B16F10 mouse model compared to the untargeted complexes (FIG. 11).

It is established that polymers such as those used in drug and gene delivery have an inherent general toxicity which can lead to cell death. This has been regarded as a potential problem and disadvantage which could impede the use of these molecules as delivery agents. A commonly made observation is that cells in tissue culture assays will display signs of apoptosis such as rounding off and reduction and loss of attachment to the tissue culture plate.

While many compounds exhibit toxicity in cytotoxicity assays this does not identify them as potential therapeutics. The key properties which distinguish a generally toxic substance from a therapeutic agent are the specificity of its action and the specificity and selectivity of its toxic effect. Our data (e.g. FIGS. 1, 2) demonstrate that the cationic polymers can exert a cytostatic effect on tumour cell lines in vitro and therapeutic effects on tumours in vivo without systemic toxicity.

In vitro tissue culture testing of compounds frequently involves tumour derived cell lines or transformed cell lines because of their favourable growth characteristics which allow facile manipulation. As a consequence it is not normally obvious to what extent a compound has specificity for diseased cells in contrast to healthy cells. An indication of potential specificity can be inferred from the differential effects specific compounds exhibit against a panel of cell lines, but the key data which demonstrates therapeutic potential is activity in animal models of cancer, such as murine tumour xenografts, as shown here.

We have previously recognised that the lower generation polypropylenimine dendrimers are synthetic transfection agents that mediate efficient transgene expression in vitro [36] and after systemic injection do not demonstrate any gross toxicity [54]. When such systems are administered in vivo in tumour bearing animals, however, the therapeutic effect seen in various tumour models is at least as good as that of doxorubicin without the systemic toxicity seen by such cytotoxic drugs.

### REFERENCES

1. Boyle, P., A. d'Onofrio, P. Maisonneuve, G. Severi, C. Robertson, M. Tubiana, and U. Veronesi, Measuring progress against cancer in Europe: has the 15% decline targeted for 2000 come about? Ann Oncol, 2003. 14(8): p. 1312-25.
2. Rodu, B. and P. Cole, The fifty-year decline of cancer in America. J Clin Oncol, 2001. 19(1): p. 239-41.
3. Schipper, R. G., L. C. Penning, and A. A. Verhofstad, Involvement of polyamines in apoptosis. Facts and controversies: effectors or protectors? Semin Cancer Biol, 2000. 10(1): p. 55-68.
4. Schipper, R. G., G. Deli, P. Deloyer, W. P. Lange, J. A. Schalken, and A. A. Verhofstad, Antitumor activity of the polyamine analog N(1),N(11)-diethylnorspermine against human prostate carcinoma cells. Prostate, 2000. 44(4): p. 313-21.
5. Reddy, V. K., A. Valasinas, A. Sarkar, H. S. Basu, L. J. Marton, and B. Frydman, Conformationally restricted analogues of 1N,12N-bisethylspermine: synthesis and growth inhibitory effects on human tumor cell lines. J Med Chem, 1998. 41(24): p. 4723-32.
6. Valasinas, A., A. Sarkar, V. K. Reddy, L. J. Marton, H. S. Basu, and B. Frydman, Conformationally restricted analogues of 1N,14N-bisethylhomospermine (BE-4-4-4): synthesis and growth inhibitory effects on human prostate cancer cells. J Med Chem, 2001. 44(3): p. 390-403.
7. Reddy, V. K., A. Sarkar, A. Valasinas, L. J. Marton, H. S. Basu, and B. Frydman, cis-Unsaturated analogues of 3,8,13,18,23-pentaazapentacosane (BE-4-4-4-4): synthesis and growth inhibitory effects on human prostate cancer cell lines. J Med Chem, 2001. 44(3): p. 404-17.
8. Devereux, W., Y. Wang, T. M. Stewart, A. Hacker, R. Smith, B. Frydman, A. L. Valasinas, V. K. Reddy, L. J. Marton, T. D. Ward, P. M. Woster, and R. A. Casero, Induction of the PAOhl/SMO polyamine oxidase by polyamine analogues in human lung carcinoma cells. Cancer Chemother Pharmacol, 2003. 52(5): p. 383-90.
9. Valasinas, A., V. K. Reddy, A. V. Blokhin, H. S. Basu, S. Bhattacharya, A. Sarkar, L. J. Marton, and B. Frydman, Long-chain polyamines (oligoamines) exhibit strong cytotoxicities against human prostate cancer cells. Bioorg Med Chem, 2003. 11(18): p. 4121-31.
10. Frydman, B., C. W. Porter, Y. Maxuitenko, A. Sarkar, S. Bhattacharya, A. Valasinas, V. K. Reddy, N. Kisiel, L. J. Marton, and H. S. Basu, A novel polyamine analog (SL-11093) inhibits growth of human prostate tumor xenografts in nude mice. Cancer Chemother Pharmacol, 2003. 51(6): p. 488-92.
11. Frydman, B., A. V. Blokhin, S. Brummel, G. Wilding, Y. Maxuitenko, A. Sarkar, S. Bhattacharya, D. Church, V. K. Reddy, J. A. Kink, L. J. Marton, A. Valasinas, and H. S. Basu, Cyclopropane-containing polyamine analogues are efficient growth inhibitors of a human prostate tumor xenograft in nude mice. J Med Chem, 2003. 46(21): p. 4586-600.
12. Frydman, B., S. Bhattacharya, A. Sarkar, K. Drandarov, S. Chesnov, A. Guggisberg, K. Popaj, S. Sergeyev, A. Yurdakul, M. Hesse, H. S. Basu, and L. J. Marton, Macrocyclic polyamines deplete cellular ATP levels and inhibit cell growth in human prostate cancer cells. J Med Chem, 2004. 47(4): p. 1051-9.
13. Liu, D., Z. Shriver, Y. Qi, G. Venkataraman, and R. Sasisekharan, Dynamic regulation of tumor growth and metastasis by heparan sulfate glycosaminoglycans. Semin Thromb Hemost, 2002. 28(1): p. 67-78.
14. Berry, D., D. M. Lynn, R. Sasisekharan, and R. Langer, Poly(beta-amino ester)s promote cellular uptake of heparin and cancer cell death. Chem Biol, 2004. 11(4): p. 487-98.
15. Ishida, K., M. K. Wierzba, T. Teruya, S. Simizu, and H. Osada, Novel heparan sulfate mimetic compounds as antitumor agents. Chem Biol, 2004. 11(3): p. 367-77.
16. Szoka, F. C., Jr. and J. Haensler, Self-assembling polynucleotide delivery system comprising dendrimer polycations, in U. S. Pat. No. 5,990,089. 1999, The Regents of the University of California: US.
17. Tomalia, D. A., J. J. R. Baker, R. C. Cheng, A. U. Bielinska, M. J. Fazio, D. M. Hedstrand, J. A. Johnson, D. A. Kaplan, S. L. Klakamp, W. J. Kruper, J. Kukowska-Latallo, B. D. Maxon, L. T. Piehler, I. A. Tomlinson, L. R. Wilson, R. Yin, I. Brothers, and H. M., Bioactive and/or targeted dendrimer conjugates, in U. S. Pat. No. 5,714,166. 1998, The Dow Chemical Company (Midland, Mich.); Dendritech Incorporated (Midland, Mich.); The Regents of the University of Michigan (Ann Arbor, Mich.).
18. Tomalia, D. A., W. J. Kruper, R. C. Cheng, I. A. Tomlinson, M. J. Fazio, D. M. Hedstrand, L. R. Wilson, and D. A. Kaplan, Starburst conjugates, in U. S. Pat. No. 6,177,414. 2001, Dow Chemical Company.
19. Yin, R., D. A. Tomalia, D. M. Hedstrand, D. R. Swanson, J. J. R. Baker, and J. F. Kukowska-Latallo, Hyper comb-branched polymer conjugates, in U. S. Pat. No. 5,919,442. 1996, Dendritech Inc., University of Michigan: US.
20. Kukowska-Latallo, J. F., A. U. Bielinska, J. Johnson, R. Spindler, D. A. Tomalia, and J. R. Baker, Efficient Transfer of Genetic Material Into Mammalian-Cells Using Starburst Polyamidoamine Dendrimers. Proceedings of the National Academy of Sciences of the United States of America, 1996. 93(10): p. 4897-4902.
21. Tang, M. X., C. T. Redemann, and F. C. Szoka, In vitro gene delivery by degraded polyamidoamine dendrimers. Bioconjugate Chemistry, 1996. 7(6): p. 703-14.
22. Hudde, T., S. A. Rayner, R. M. Corner, M. Weber, J. D. Isaacs, H. Waldmann, D. F. Larkin, and A. J. George, Activated polyamidoamine dendrimers, a non-viral vector for gene transfer to the corneal endothelium. Gene Therapy, 1999. 6(5): p. 939-43.
23. Bielinska, A. U., C. L. Chen, J. Johnson, and J. R. Baker, DNA complexing with polyamidoamine dendrimers: Implications for transfection. Bioconjugate Chemistry, 1999. 10(5): p. 843-850.
24. Bielinska, A. U., J. F. Kukowska-Latallo, and J. R. Baker, The interaction of plasmid DNA with polyamidoamine dendrimers: mechanism of complex formation and analysis of alterations induced in nuclease sensitivity and transcriptional activity of the complexed DNA. Biochim Biophys Acta, 1997. 1353(2): p. 180-90.
25. Bielinska, A. U., A. Yen, H. L. Wu, K. M. Zahos, R. Sun, N. D. Weiner, J. R. Baker, and B. J. Roessler, Application of membrane-based dendrimer/DNA complexes for solid phase transfection in vitro and in vivo. Biomaterials, 2000. 21(9): p. 877-87.
26. Toth, I., T. Sakthivel, A. F. Wilderspin, H. Bayele, M. O'Donnell, D. J. Perry, K. J. Pasi, C. A. Lee, and A. T. Florence, Novel cationic lipidic peptide dendrimer vectors—In vitro gene delivery. STP Pharma Sci., 1999. 9(1): p. 93-99.
27. Du, B., R. J. Zhou, and R. X. Zhuo, Synthesis of cyclic core dendritic polymer and its usage as a vector for transferring foreign DNA into human cells. Chin. Chem. Lett., 1998. 9(7): p. 635-638.
28. Loup, C., M. A. Zanta, A. M. Caminade, J. P. Majoral, and B. Meunier, Preparation of water soluble cationic phosphorus containing dendrimers as DNA transfecting agents. Chemistry a European Journal, 1999. 5: p. 3644-3650.
29. Pistolis, G., A. Malliaris, D. Tsiourvas, and C. M. Paleos, Poly(propyleneimine) dendrimers as pH-sensitive controlled-release systems. Chem.-Eur. J., 1999. 5(5): p. 1440-1444.
30. Sideratou, Z., D. Tsiourvas, and C. M. Paleos, Quaternized poly(propylene imine) dendrimers as novel pH-sensitive controlled-release systems. Langmuir, 2000. 16(4): p. 1766-1769.
31. Jansen, J., E. M. M. Debrabandervandenberg, and E. W. Meijer, Encapsulation of Guest Molecules into a Dendritic Box. Science, 1994. 266(5188): p. 1226-1229.
32. Kobayashi, H. and M. Brechbiel, Macromolecular Imaging Agents for Liver Imaging, in WO03001218 A2. 2003.
33. Kabanov, V. A., A. B. Zezin, V. B. Rogacheva, Z. G. Gulyaeva, M. F. Zansochova, J. G. H. Joosten, and J. Brackman, Interaction of Astramol poly(propyleneimine) dendrimers with linear polyanions. Macromolecules, 1999. 32(6): p. 1904-1909.
34. Gebhart, C. L. and A. V. Kabanov, Evaluation of polyplexes as gene transfer agents. J Control Release, 2001. 73(2-3): p. 401-16.
35. Malik, N., R. Wiwattanapatapee, R. Klopsch, K. Lorenz, H. Frey, J. W. Weener, E. W. Meijer, W. Paulus, and R. Duncan, Dendrimers: Relationship between structure and biocompatibility in vitro, and preliminary studies on the biodistribution of I-125-labelled polyamidoamine dendrimers in vivo. J. Control. Release, 2000. 65(1-2): p. 133-148.
36. Zinselmeyer, B. H., S. P. Mackay, A. G. Schatzlein, and I. F. Uchegbu, The lower-generation polypropylenimine dendrimers are effective gene-transfer agents. Pharm Res, 2002. 19(7): p. 960-7.
37. Uchegbu, I. F., A. G. Schatzlein, A. I. Gray, B. Zinselmeyer, and A. Munro, Polypropylenimine dendrimers for gene delivery. 2002: GB.
38. Hollins, A. J., M. Benboubetra, Y. Omidi, B. H. Zinselmeyer, A. G. Schatzlein, I. F. Uchegbu, and S. Akhtar, Evaluation of generation 2 and 3 poly(propylenimine) dendrimers for the potential cellular delivery of antisense oligonucleotides targeting the epidermal growth factor receptor. Pharm Res, 2004. 21(3): p. 458-66.
39. Wiwattanapatapee, R., B. Carreno-Gomez, N. Malik, and R. Duncan, Anionic PAMAM dendrimers rapidly cross adult rat intestine in vitro: a potential oral delivery system? Pharm Res, 2000. 17(8): p. 991-8.
40. Boussif, O., F. Lezoualc'h, M. A. Zanta, M. D. Mergny, D. Scherman, B. Demeneix, and J. P. Behr, A versatile vector for gene and oligonucleotide transfer into cells in culture and in vivo: polyethylenimine. Proc. Natl. Acad. Sci. USA, 1995. 92(16): p. 7297-301.
41. Godbey, W. T., K. K. Wu, and A. G. Mikos, Size matters: Molecular weight affects the efficiency of poly(ethylenimine) as a gene delivery vehicle. J. Biomed. Mater. Res., 1999. 45(3): p. 268-275.
42. Fischer, D., T. Bieber, Y. Li, H. P. Elsasser, and T. Kissel, A novel non-viral vector for DNA delivery based on low molecular weight, branched polyethylenimine: effect of molecular weight on transfection efficiency and cytotoxicity. Pharm. Res., 1999. 16(8): p. 1273-9.
43. Ogris, M., S. Brunner, S. Schuller, R. Kircheis, and E. Wagner, PEGylated DNA/transferrin-PEI complexes: reduced interaction with blood components, extended circulation in blood and potential for systemic gene delivery. Gene Ther, 1999. 6(4): p. 595-605.
44. Goula, D., C. Benoist, S. Mantero, G. Merlo, G. Levi, and B. A. Demeneix, Polyethylenimine-based intravenous delivery of transgenes to mouse lung. Gene Ther, 1998. 5(9): p. 1291-5.
45. Kunath, K., A. von Harpe, D. Fischer, H. Petersen, U. Bickel, K. Voigt, and T. Kissel, Low-molecular-weight polyethylenimine as a non-viral vector for DNA delivery: comparison of physicochemical properties, transfection efficiency and in vivo distribution with high-molecular-weight polyethylenimine. J Control Release, 2003. 89(1): p. 113-25.
46. Goula, D., J. S. Remy, P. Erbacher, M. Wasowicz, G. Levi, B. Abdallah, and B. A. Demeneix, Size, diffusibility and transfection performance of linear PEI/DNA complexes in the mouse central nervous system. Gene Ther, 1998. 5(5): p. 712-7.
47. Bragonzi, A., A. Boletta, A. Biffi, A. Muggia, G. Sersale, S. H. Cheng, C. Bordignon, B. M. Assael, and M. Conese, Comparison between cationic polymers and lipids in mediating systemic gene delivery to the lungs. Gene Ther, 1999. 6(12): p. 1995-2004.
48. Abdallah, B., A. Hassan, C. Benoist, D. Goula, J. P. Behr, and B. A. Demeneix, A powerful nonviral vector for in vivo gene transfer into the adult mammalian brain: polyethylenimine. Hum Gene Ther, 1996. 7(16): p. 1947-54.
49. Li, S., Y. Tan, E. Viroonchatapan, B. R. Pitt, and L. Huang, Targeted gene delivery to pulmonary endothelium by anti-PECAM antibody. Am J Physiol, 2000. 278(3): p. L504-L511.
50. Furgeson, D. Y., R. N. Cohen, R. I. Mahato, and S. W. Kim, Novel water insoluble lipoparticulates for gene delivery. Pharm Res, 2002. 19(4): p. 382-90.
51. Han, S., R. I. Mahato, and S. W. Kim, Water-soluble lipopolymer for gene delivery. Bioconjug. Chem., 2001. 12(3): p. 337-45.
52. Kircheis, R., L. Wightman, M. Kursa, E. Ostermann, and E. Wagner, Tumor-targeted gene delivery: an attractive strategy to use highly active effector molecules in cancer treatment. Gene Therapy, 2002. 9(11): p. 731-735.
53. Brownlie, A., I. F. Uchegbu, and A. G. Schatzlein, PEI-based vesicle-polymer hybrid gene delivery system with improved biocompatibility. Int J Pharm, 2004. 274(1-2): p. 41-52.
54. Zinselmeyer, B. H., N. Beggbie, I. F. Uchegbu, and A. G. Schatzlein, Quantification of beta-galactosidase activity after non-viral transfection in vivo. J Control Release, 2003. 91(1-2): p. 201-8.
55. Dufes, C., J. M. Muller, W. Couet, J. C. Olivier, I. F. Uchegbu, and A. G. Schatzlein, Anticancer drug delivery with transferrin targeted polymeric chitosan vesicles. Pharm Res, 2004. 21(1): p. 101-7.
56. Al-Hajj, M., M. S. Wicha, A. Benito-Hernandez, S. J. Morrison, and M. F. Clarke, Prospective identification of tumorigenic breast cancer cells. Proc Natl Acad Sci USA, 2003. 100(7): p. 3983-8.
57. Schätzlein, A. G. In Vivo Gene Delivery with Polypropylenimine Dendrimer Systems. in Genes as Medicines. 2004. London, UK: The Academy of Pharmaceutical Sciences.
58. Elouzi, A., A. I. Gray, I. F. Uchegbu, and A. G. Schatzlein. Tumour Targeting of Gene Expression Using Hyaluronic Acid-Polypropylenimine Dendrimer Conjugates. in 30th Annual Meeting & Exposition of the Controlled Release Society. 2003. Glasgow, UK: Controlled Release Soviety.
59. Shaunak, S., S. Thomas, E. Gianasi, A. Godwin, E. jones, I. Teo, K. Mireskandari, P. Luthert, R. Duncan, S. Patterson, P. Khaw, and S. Brocchini, Polyvalent dendrimer glucosamine conjugates prevent scar tissue formation. Nature Biotechnology, Vol. 22, No. 8, August 2004, p. 977-984.
60. Gong, Y., B. Matthews, D. Cheung, T. Tam, I. Gadawski, D. Leung, G. Holan, J. Raff, S. Sacks, Evidence of dual sites of action of dendrimers: SPL-2999 inhibits both virus entry and late stages of herpes simplex virus replication. Antiviral Research 55 (2002) 319-329

1. A method for the treatment of a condition characterised by undesirable cellular proliferation in a patient in need of said treatment, said method comprising administering to said patient a therapeutically effective amount of a dendrimer compound of the general formula IV or a salt thereof: 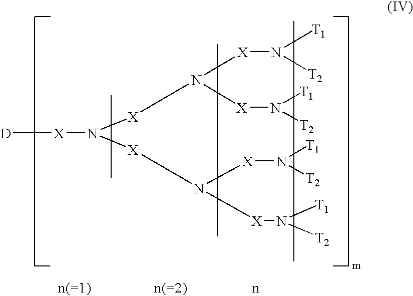
wherein
n is greater than or equal to 1, wherein n represents the number of generations of the dendrimer;
D is a core group of the dendrimer including a plurality of functional atoms;
X is selected from optionally substituted C₁₋₁₆ alkylene groups independently for each generation of the dendrimer, wherein said C₁₋₁₆ alkylene groups are independently optionally interrupted by one or more N(R²) or O heterogroups wherein each R² is independently H or optionally substituted C₁₋₁₆ alkyl optionally interrupted by one or more N(R²) or O heterogroups;
m is an integer from 2 to 8, wherein m denotes the number of X groups of the first generation that are bonded to the core group, wherein each X group of the first generation is bonded to a core functional atom; and
T₁ and T₂ represent end groups bonded to the nth generation of the dendrimer, wherein T₁ and T₂ are independently selected from the substituents defined herein.

2. The method according to claim 1 wherein said C₁₋₁₆ alkyl and C₁₋₁₆ alkylene groups are optionally substituted by one or more groups selected from oxo, amino, hydroxy, carboxy, alkoxy, ester and halo.

3. (canceled)

4. The method according to claim 1 wherein T₁ and T₂ are independently selected from H, hydroxy, carboxy, halo and optionally substituted amino, amido, alkoxy, acyl, ester, C₁₋₁₆ alkyl, C₃₋₇ heterocyclyl, C₅₋₁₀ aryl, C₅₋₁₀ heteroaryl, C₁₋₁₆ alkylene-NR³R⁴, C₅₋₁₀ arylene-NR³R⁴, C₁₋₁₆ alkylene-C₅₋₁₀ arylene-NR³R⁴, and C₅₋₁₀ arylene-C₁₋₁₆ alkylene-NR³R⁴, wherein R³ and R⁴ are independently selected from H and optionally substituted C₁₋₁₆ alkyl and C₅₋₁₀ aryl, wherein said C₁₋₁₆ alkyl and C₁₋₁₆ alkylene groups are optionally interrupted by one or more N(R²) or O heterogroups.

5-11. (canceled)

12. The method according to claim 1 wherein D is selected from: 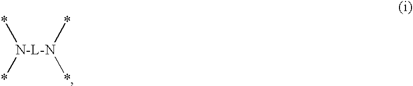
wherein m is 4 and L is C₁₋₁₆ alkylene; 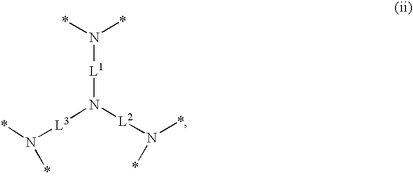
wherein m is 6 and L¹, L² and L³ are independently selected from C₁₋₁₆ alkylene groups; 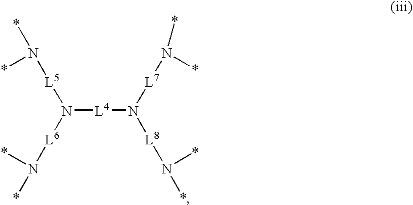
wherein m is 8 and L⁴, L⁵, L⁶, L⁷ and L⁸ are independently selected from C₁₋₁₆ alkylene groups; and 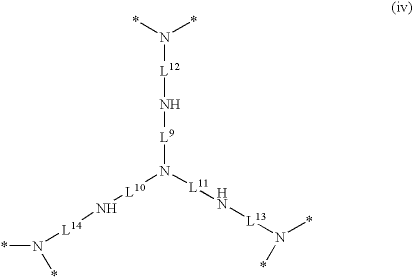
wherein m is 6; L⁹, L¹⁰ and L¹¹ are independently selected from C₁₋₄ alkyl groups; and L¹², L¹³ and L¹⁴ are independently selected from C₁₋₁₆ alkylene groups;
wherein * represents a point of covalent attachment to an X group of the first generation, and wherein each of said C₁₋₁₆ alkylene groups is optionally interrupted by one or more N(R²) or O heterogroups and optionally substituted by one or more groups selected from oxo, amino, hydroxy, carboxy, alkoxy, ester and halo.

13. (canceled)

14. The method according to claim 1 wherein D is 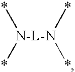
m is 4 and L is ethylene, propylene, butylene, hexylene or dodecylene.

15. (canceled)

16. The method according to claim 1 wherein D is 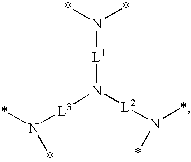
m is 6 and L¹, L², and L³ are selected from groups having the general structure Cₚ alkylene-C(O)N(R²)—C_{q} alkylene wherein p and q are integers and p+q is in the range 2 to 16.

17. (canceled)

18. The method according to claim 1 wherein D is 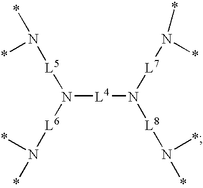
m is 8;
L⁴ is a linear unsubstituted C₁₋₁₂ alkylene group; and
L⁵, L⁶, L⁷ and L⁸ are selected from groups having the general structure Cₚ alkylene-C(O)N(R²)—C_{q} alkylene wherein p and q are integers and p+q is in the range 2 to 16.

19. (canceled)

20. (canceled)

21. The method according to claim wherein D is 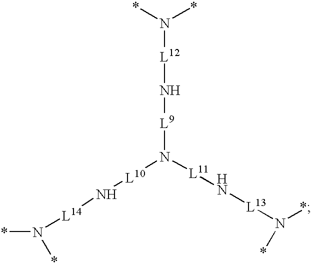
wherein
m is 6;
L⁹, L¹⁰ and L¹¹ are linear unsubstituted C₁₋₄ alkylene groups; and
L¹², L¹³ and L¹⁴ are selected from groups having the general structure Cₚ alkylene-C(O)N(R²)—C_{q} alkylene wherein p and q are integers and p+q is in the range 2 to 16.

22. (canceled)

23. (canceled)

24. (canceled)

25. The method according to claim 1 wherein X is selected from groups having the general structure Cₚ alkylene-C(O)N(R²)—C_{q} alkylene wherein p and q are integers and p+q is in the range 2 to 16.

26. The method according to claim 1 wherein X is selected from groups having the general structure C₁₋₆ alkylene-C(O)NH—C₁₋₆ alkylene.

27. The method according to claim 1 wherein X is selected from linear unsubstituted C₁₋₁₆ alkylene groups.

28. (canceled)

29. (canceled)

30. The method according to claim 29 wherein X is —(CH₂)₂—C(═O)N(H)—(CH₂)₂—.

31. (canceled)

32. The method according to claim 1 wherein T₁ and T₂ are both H or C₁₋₄ alkyl, so that the terminal groups of the dendrimer are NH₂ or N(R⁵)₂ wherein R⁵ is C₁₋₄ alkyl.

33. The method according to claim 1 wherein T₁ and T₂ are both H or methyl, so that the terminal groups of the dendrimer are either NH₂ or NMe₂.

34-47. (canceled)

48. of a compound of formula I or a salt thereof as an active agent in the preparation of a medicament A method for the treatment of a condition characterised by undesirable cellular proliferation in a patient in need of said treatment, said method comprising administering to said patient a therapeutically effective amount of a compound of formula I or a salt thereof: 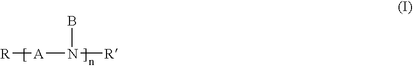
wherein
R is independently selected from H, optionally substituted C₁₋₁₆ alkyl and NR²R³ wherein R² and R³ are independently selected from H and optionally substituted C₁₋₁₆ alkyl;
R′ is independently selected from H and optionally substituted C₁₋₁₆ alkyl;
n denotes the number of backbone monomer units -[A-N(B)]— and is greater than or equal to 15;
the A groups of the backbone monomer units are independently selected from optionally substituted C₁₋₁₆ alkylene groups; and
the B groups of the backbone monomer units are independently selected from H, optionally substituted C116 alkyl and a branching group of formula II: 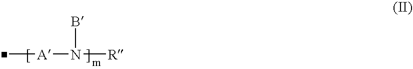
wherein
R″ is selected from H, optionally substituted C₁₋₁₆ alkyl and optionally substituted C₁₋₁₆ alkylene-NR²R³;
m denotes the number of monomer units -[A′-N(B′)]— of the branching group and is greater than or equal to 1;
the A′ groups of the monomer units of the branching group are independently selected from optionally substituted C₁₋₁₆ alkylene groups; and
the B′ groups of the monomer units of the branching group are independently selected from H, optionally substituted C₁₋₁₆ alkyl and a branching group of formula II;
wherein each of said C₁₋₁₆ alkyl and C₁₋₁₆ alkylene groups is optionally interrupted by one or more N(R²) or O heterogroups.

49-70. (canceled)

71. The method according to claim 48 wherein the branching groups of formula II are located on average, at every qth nitrogen atom along any given polymer chain segment, wherein q is greater than 3.

72-80. (canceled)

81. The method according to claim 48 wherein the compound of formula I is associated with a targeting moiety.

82-89. (canceled)

90. A composition for delivering a bioactive molecule other than a nucleic acid to a target location in vivo, the composition comprising a compound of formula I as defined in claim 48,
except that n, which denotes the number of backbone monomer units -[A-N(B)]—, is greater than or equal to 3,
or a salt thereof admixed with said bioactive molecule, wherein the composition does not contain nucleic acid.

91-93. (canceled)

94. in the preparation of a medicament A method for the treatment of a condition characterised by undesirable cellular proliferation in a patient in need of said treatment, said method comprising administering to said patient a therapeutically effective amount of a composition as claimed in claim 90.

95. (canceled)

96. The method according to claim 48 wherein the dendrimeter compound of formula IV is associated with a target moiety.
